(19) United States
(12) Patent Application Publication
(10) Pub. No.: US 2022/0227763 A1
(43) Pub. Date: Jul. 21, 2022

(54) MICROBIOCIDAL DERIVATIVES

(71) Applicant: SYNGENTA CROP PROTECTION AG, Basel (CH)

(72) Inventors: Mattia Riccardo MONACO, Stein (CH); Stefano RENDINE, Stein (CH); Daniel STIERLI, Stein (CH); Mathias BLUM, Stein (CH); Andrew EDMUNDS, Stein (CH)

(73) Assignee: SYNGENTA CROP PROTECTION AG, Basel (CH)

(21) Appl. No.: 17/613,305

(22) PCT Filed: May 27, 2020
(86) PCT No.: PCT/EP2020/064736

§ 371 Date: Nov. 22, 2021

(30) Foreign Application Priority Data
May 29, 2019 (EP) 19177421.5

(51) Int. Cl. C07D 471/04; A01N 43/90; A01P 3/00

(52) U.S. Cl. CPC C07D 471/04; A01P 3/00; A01N 43/90

(57) ABSTRACT

Compounds of the formula (I) wherein the substituents are as defined in claim 1, useful as pesticides, and especially fungicides.

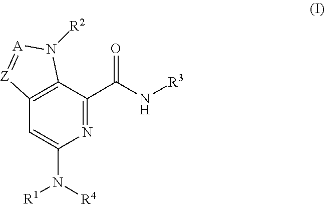

The present invention relates to microbiocidal azaindole and azaindazole derivatives, e.g., as active ingredients, which have microbiocidal activity, in particular fungicidal activity. The invention also relates to the preparation of these azaindole and azaindazole derivatives, to agrochemical compositions which comprise at least one of the azaindole or azaindazole derivatives and to uses of the azaindole and azaindazole derivatives or compositions thereof in agriculture or horticulture for controlling or preventing the infestation of plants, harvested food crops, seeds or non-living materials by phytopathogenic microorganisms, preferably fungi.

WO 2015/040405 describes pyridinecarboxamide derivatives as pesticidal agents.

According to the present invention, there is provided a compound of formula (I):

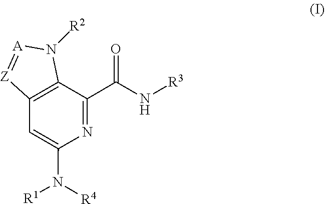

wherein

A is N or C—R⁵;

Z is N or C—R⁵;

R¹ is hydrogen, cyano, formyl, C₁-C₆alkylcarbonyl, C₁-C₆alkoxycarbonyl, C₁-C₆haloalkylcarbonyl, C₁-C₆alkoxyC₁-C₆alkylcarbonyl, C₃-C₆cycloalkylcarbonyl, C₁-C₆alkoxyC₁-C₃alkoxycarbonyl, C₁-C₆alkoxyoxalyl, C₁-C₆alkoxycarbonylC₁-C₄alkylC₁-C₆alkoxycarbonyl, C₁-C₆alkylsulfanylcarbonyl, or phenylcarbonyl;

R² is hydrogen, C₁-C₆alkyl, C₁-C₆haloalkyl, cyano, formyl, C₁-C₆alkylcarbonyl, C₁-C₆alkoxycarbonyl, C₁-C₆haloalkylcarbonyl, C₁-C₆alkoxyC₁-C₆alkylcarbonyl, C₃-C₆cycloalkylcarbonyl, C₁-C₆alkoxyC₁-C₃alkoxycarbonyl, C₁-C₆alkoxyoxalyl, C₁-C₆alkoxycarbonylC₁-C₄alkylC₁-C₆alkoxycarbonyl, C₂-C₆alkenyloxycarbonyl, C₂-C₆alkynyloxycarbonyl, C₁-C₆alkylsulfanylcarbonyl, or phenylcarbonyl;

R³ is C₁-C₈alkyl, C₁-C₈haloalkyl, C₁-C₈alkoxy, C₃-C₈cycloalkyl, C₃-C₈cycloalkylC₁-C₂alkyl, wherein the cycloalkyl groups are optionally substituted with 1 to 3 groups represented by R⁶, phenyl, phenylC₁-C₂alkyl, heteroaryl, heteroarylC₁-C₂alkyl, wherein the heteroaryl is a 5-or 6-membered aromatic monocyclic ring comprising 1, 2, 3 or 4 heteroatoms individually selected from nitrogen, oxygen and sulfur, heterocyclyl, heterocyclylC₁-C₂alkyl, wherein the heterocyclyl is a 4-, 5-or 6-membered non-aromatic monocyclic ring comprising 1, 2 or 3 heteroatoms individually selected from nitrogen, oxygen and sulfur, or a 5-to 10-membered non-aromatic spirocyclic carbobi- or carbotri-cyclyl ring system optionally comprising 1, 2, 3, 4 or 5 heteroatoms individually selected from nitrogen, oxygen and sulfur, and optionally bonded to the rest of the molecule through a C₁-C₂alkylene linker;

R⁴ is phenyl or heteroaryl, wherein heteroaryl is a 5-or 6-membered aromatic monocyclic ring comprising 1, 2 or 3 heteroatoms individually selected from nitrogen, oxygen and sulfur, and wherein the phenyl or heteroaryl group is optionally substituted by 1, 2 or 3 substituents, which may be the same or different, selected from R⁷;

R⁵ is hydrogen, halogen, or C₁-C₄alkyl;

R⁶ is halogen, C₁-C₄alkyl, C₁-C₄alkoxy, or C₁-C₄haloalkyl; and

R⁷ is halogen, cyano, C₁-C₄alkyl, C₁-C₄haloalkyl, C₁-C₄alkoxy, or C₁-C₄haloalkoxy;

or a salt or an N-oxide thereof.

Surprisingly, it has been found that the novel compounds of formula (I) have, for practical purposes, a very advantageous level of biological activity for protecting plants against diseases that are caused by fungi.

According to a second aspect of the invention, there is provided an agrochemical composition comprising a fungicidally effective amount of a compound of formula (I) according to the present invention. Such an agricultural composition may further comprise at least one additional active ingredient and/or an agrochemically-acceptable diluent or carrier.

According to a third aspect of the invention, there is provided a method of controlling or preventing infestation of useful plants by phytopathogenic microorganisms, wherein a fungicidally effective amount of a compound of formula (I), or a composition comprising this compound as active ingredient, is applied to the plants, to parts thereof or the locus thereof.

According to a fourth aspect of the invention, there is provided the use of a compound of formula (I) as a fungicide. According to this particular aspect of the invention, the use may or may not include methods for the treatment of the human or animal body by surgery or therapy.

Where substituents are indicated as being “optionally substituted”, this means that they may or may not carry one or more identical or different substituents, e.g., one, two or three R⁶ substituents. For example, C₁-C₈alkyl substituted by 1, 2 or 3 halogens, may include, but not be limited to, —CH₂Cl, —CHCl₂, —CCl₃, —CH₂F, —CHF₂, —CF₃, —CH₂CF₃ or —CF₂CH₃ groups. As another example, C₁-C₆alkoxy substituted by 1, 2 or 3 halogens, may include, but not limited to, CH₂ClO—, CHCl₂O—, CCl₃O—, CH₂FO—, CHF₂O—, CF₃O—, CF₃CH₂O— or CH₃CF₂O— groups.

As used herein, the term “cyano” means a —CN group.

As used herein, the term “halogen” refers to fluorine (fluoro), chlorine (chloro), bromine (bromo) or iodine (iodo).

As used herein, the term “formyl” means a —C(O)H group.

As used herein, the term “acetyl” means a —C(O)CH₃ group.

As used herein, the term “C₁-C₈alkyl” refers to a straight or branched hydrocarbon chain radical consisting solely of carbon and hydrogen atoms, containing no unsaturation, having from one to eight carbon atoms, and which is attached to the rest of the molecule by a single bond. “C₁-C₆alkyl”, “C₁-C₄alkyl” and “C₁-C₃alkyl” are to be construed accordingly. Examples of C₁-C₈alkyl include, but are not limited to, methyl, ethyl, n-propyl, and the isomers thereof, for example, iso-propyl. A “C₁-C₆alkylene” group refers to the corresponding definition of C₁-C₆alkyl, except that such radical is attached to the rest of the molecule by two single bonds. The term “C₁-C₂alkylene” is to be construed accordingly. Examples of C₁-C₆alkylene, include, but are not limited to, —CH₂—, —CH₂CH₂— and —(CH₂)₃—.

As used herein, the term “C₁-C₈haloalkyl” refers a C₁-C₈alkyl radical as generally defined above substituted by one or more of the same or different halogen atoms. The terms “C₁-C₆haloalkyl” and “C₁-C₄haloalkyl”, are to be construed accordingly. Examples of C₁-C₈haloalkyl include, but are not limited to trifluoromethyl.

As used herein, the term “C₁-C₈alkoxy” refers to a radical of the formula —ORₐ where Ra is a C₁-C₈alkyl radical as generally defined above. The terms “C₁-C₆alkoxy”, “C₁-C₄alkoxy” and “C₁-C₃alkoxy” are to be construed accordingly. Examples of C₁-C₈alkoxy include, but are not limited to, methoxy, ethoxy, 1-methylethoxy (iso-propoxy), and propoxy.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. The term “C₂-C₃alkenyl” is to be construed accordingly. Examples of C₂-C₆alkenyl include, but are not limited to, ethenyl (vinyl), prop-1-enyl, prop-2-enyl (allyl), but-1-enyl.

As used herein, the term “C₂-C₆alkenyloxy” refers to a radical of the formula —ORₐ where Rₐ is a C₂-C₈alkenyl radical as generally defined above.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. The term “C₂-C₃alkynyl” is to be construed accordingly. Examples of C₂-C₆alkynyl include, but are not limited to, ethynyl, prop-1-ynyl, but-1-ynyl.

As used herein, the term “C₂-C₆alkynyloxy” refers to a radical of the formula —ORₐ where Rₐ is a C₂-C₈alkynyl radical as generally defined above.

As used herein, the term “C₃-C₈cycloalkyl” refers to a radical which is a monocyclic saturated ring system and which contains 3 to 8 carbon atoms. The terms “C₃-C₆cycloalkyl”, “C₃-C₄cycloalkyl” are to be construed accordingly. Examples of C₃-C₆cycloalkyl include, but are not limited to, cyclopropyl, 1-methylcyclopropyl, 2-methylcyclopropyl, cyclobutyl, 1-methylcyclobutyl, 1,1-dimethylcyclobutyl, 2-methylcyclobutyl, and 2,2-dimethylcyclobutyl.

As used herein, the term “C₃-C₈cycloalkylC₁-C₂alkyl” refers to a C₃-C₈cycloalkyl ring attached to the rest of the molecule by a C₁-C₂alkylene linker as defined above.

As used herein, the term “phenylC₁-C₂alkyl” refers to a phenyl ring attached to the rest of the molecule by a C₁-C₂alkylene linker as defined above.

As used herein, the term “C₁-C₆alkoxyoxalyl”, refers to —C(O)C(O)ORₐ radical, where Rₐ C₁-C₆alkyl radical as generally defined above.

As used herein, the term “heteroaryl” refers to a 5-or 6-membered aromatic monocyclic ring radical which comprises 1, 2, 3 or 4 heteroatoms individually selected from nitrogen, oxygen and sulfur. Examples of heteroaryl include, but are not limited to, furanyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl.

As used herein, the term “heteroarylC₁-C₂alkyl” refers to a heteroaryl ring attached to the rest of the molecule by a C₁-C₂alkylene linker as defined above.

As used herein, the term “heterocyclyl” refers to a stable 4-, 5-or 6-membered non-aromatic monocyclic ring which comprises 1, 2 or 3 heteroatoms, wherein the heteroatoms are individually selected from nitrogen, oxygen and sulfur. The heterocyclyl radical may be bonded to the rest of the molecule via a carbon atom or heteroatom. Examples of heterocyclyl include, but are not limited to, aziridinyl, azetidinyl, oxetanyl, thietanyl, tetrahydrofuryl, pyrrolidinyl, pyrazolidinyl, imidazolidnyl, piperidinyl, piperazinyl, morpholinyl, dioxolanyl, dithiolanyl and thiazolidinyl.

As used herein, the term “heterocyclylC₁-C₂alkyl” refers to a heterocyclyl ring attached to the rest of the molecule by a C₁-C₂alkylene linker as defined above.

As used herein, a “spirocyclic carbobi- or carbotri-cyclyl ring” is a non-aromatic bicyclic ring system comprising two rings joined together at one carbon atom, i.e., sharing one carbon atom.

Examples of a spirocyclic carbobi- or carbotri-cyclyl ring system include, but are not limited to, spiro[3.3]heptanyl, spiro[3.4]octanyl, spiro[4.5]decanyl, spiro[cyclobutan-1,2′-indanyl], or spiro[cyclopentane-1,2′-tetralinyl].

As used herein, the term “C₁-C₆alkylcarbonyl” refers to a radical of the formula —C(O)Rₐ, where Rₐ is a C₁-C₆alkyl radical as generally defined above.

As used herein, the term “C₁-C₆alkoxyC₁-C₆alkylcarbonyl” refers to a radical of the formula —C(O)RₐOR_{b}, where Rb is a C₁-C₆alkyl radical as generally defined above, and Rₐ is a C₁-C₆alkylene radical as generally defined above.

As used herein, the term “C₁-C₆haloalkylcarbonyl” refers to a radical of the formula —C(O)Rₐ, where Rₐ is a C₁-C₆haloalkyl radical as generally defined above.

As used herein, the term “C₃-C₆cycloalkylcarbonyl” refers to a radical of the formula —C(O)Rₐ, where Rₐ is a C₃-C₆cycloalkyl radical as generally defined above.

As used herein, the term “C₁-C₆alkoxycarbonyl” refers to a radical of the formula —C(O)ORₐ, where Rₐ is a C₁-C₆alkyl radical as generally defined above.

As used herein, the term “C₂-C₆alkenyloxycarbonyl” refers to a radical of the formula —C(O)ORₐ, where Rₐ is a C₂-C₆alkenyl radical as generally defined above.

As used herein, the term “C₂-C₆alkynyloxycarbonyl” refers to a radical of the formula —C(O)ORₐ, where Rₐ is a C₂-C₆alkynyl radical as generally defined above.

As used herein, the term “C₁-C₆alkylsulfanylcarbonyl” refers to a radical of the formula —C(O)SRₐ, where Rₐ is a C₁-C₆alkyl radical as generally defined above.

As used herein, the term “phenylcarbonyl” refers to a radical of the formula —C(O)Rₐ, where Rₐ is a phenyl radical.

The presence of one or more possible stereogenic elements in a compound of formula (I) means that the compounds may occur in optically isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers. The present invention includes all possible tautomeric forms for a compound of formula (I).

In each case, the compounds of formula (I) according to the invention are in free form, in oxidized form as an N-oxide, or in salt form, e.g., an agronomically usable salt form.

N-oxides are oxidized forms of tertiary amines or oxidized forms of nitrogen-containing heteroaromatic compounds. They are described for instance in the book “Heterocyclic N-oxides” by A. Albini and S. Pietra, CRC Press, Boca Raton (1991).

The following list provides definitions, including preferred definitions, for substituents R¹, R², R³, R⁴, R⁵, R⁶, R⁷, A and Z, with reference to compounds of formula (I). For any one of these substituents, any of the definitions given below may be combined with any definition of any other substituent given below or elsewhere in this document.

A is N or C—R⁵. In one set of embodiments, A is N. In another set of embodiments A is C—R⁵.

Z is N or C—R⁵. In one set of embodiments, Z is N. In another set of embodiments Z is C—R⁵.

In one set of embodiments, when A is N, Z is N or C—R⁵, wherein R⁵ is hydrogen or methyl. In another set of embodiments, when A is N, Z is C—R⁵, wherein R⁵ is hydrogen.

In a further set of embodiments, when A is C—R⁵, wherein R⁵ is hydrogen or methyl, Z is N or C—R⁵, wherein R⁵ is hydrogen or methyl. In a further still set of embodiments, when A is C—R⁵, wherein R⁵ is hydrogen or methyl, Z is C—R⁵, wherein R⁵ is hydrogen.

Preferably, A is N or CH. Preferably, Z is CH.

R¹ is hydrogen, cyano, formyl, C₁-C₆alkylcarbonyl, C₁-C₆alkoxycarbonyl, C₁-C₆haloalkylcarbonyl, C₁-C₆alkoxyC₁-C₆alkylcarbonyl, C₃-C₆cycloalkylcarbonyl, C₁-C₆alkoxyC₁-C₃alkoxycarbonyl, C₁-C₆alkoxyoxalyl, C₁-C₆alkoxycarbonylC₁-C₄alkylC₁-C₆alkoxycarbonyl, C₁-C₆alkylsulfanylcarbonyl, or phenylcarbonyl. Preferably, R¹ is hydrogen, cyano, C₁-C₆alkylcarbonyl, C₁-C₄alkoxycarbonyl, C₁-C₄haloalkylcarbonyl, C₁-C₄alkoxyC₁-C₃alkylcarbonyl, C₃-C₆cycloalkylcarbonyl, or C₁-C₄alkoxyC₁-C₂alkoxycarbonyl. More preferably, R¹ is hydrogen, cyano, or C₁-C₆alkylcarbonyl, even more preferably, hydrogen, cyano, or C₁-C₃alkylcarbonyl. More preferably still, R¹ is hydrogen, cyano, or acetyl, even more preferably, hydrogen or cyano. Most preferably, R¹ is hydrogen.

R² is hydrogen, C₁-C₆alkyl, C₁-C₆haloalkyl, cyano, formyl, C₁-C₆alkylcarbonyl, C₁-C₆alkoxycarbonyl, C₁-C₆haloalkylcarbonyl, C₁-C₆alkoxyC₁-C₆alkylcarbonyl, C₃-C₆cycloalkylcarbonyl, C₁-C₆alkoxyC₁-C₃alkoxycarbonyl, C₁-C₆alkoxyoxalyl, C₁-C₆alkoxycarbonylC₁-C₄alkylC₁-C₆alkoxycarbonyl, C₂-C₆alkenyloxycarbonyl, C₂-C₆alkynyloxycarbonyl, C₁-C₆alkylsulfanylcarbonyl, or phenylcarbonyl. Preferably, R² is hydrogen, C₁-C₆alkyl, C₁-C₆haloalkyl, cyano, formyl, C₁-C₆alkylcarbonyl, C₁-C₆alkoxycarbonyl, C₁-C₆haloalkylcarbonyl, C₁-C₆alkoxyC₁-C₆alkylcarbonyl, C₃-C₆cycloalkylcarbonyl, C₁-C₆alkoxyC₁-C₃alkoxycarbonyl, C₁-C₆alkoxyoxalyl, C₁-C₆alkoxycarbonylC₁-C₄alkylC₁-C₆alkoxycarbonyl, or phenylcarbonyl. More preferably, R² is hydrogen, C₁-C₆alkyl, C₁-C₆haloalkyl, cyano, C₁-C₄alkylcarbonyl, C₁-C₄alkoxycarbonyl, C₁-C₄alkoxyC₁-C₃alkylcarbonyl, C₁-C₄alkoxyC₁-C₃alkoxycarbonyl, C₁-C₄alkoxyoxalyl, or C₁-C₄alkoxycarbonylC₁-C₃alkylC₁-C₃alkoxycarbonyl. Even more preferably, R² is hydrogen, C₁-C₆alkyl, C₁-C₆haloalkyl, cyano, C₁-C₄alkylcarbonyl, C₁-C₆haloalkyl, cyano, C₁-C₄alkylcarbonyl, C₁-C₄alkoxycarbonyl. More preferably still, R² is hydrogen, C₁-C₄alkyl, or C₁-C₄alkylcarbonyl, even more preferably, hydrogen, methyl, ethyl, isopropyl, acetyl or ethylcarbonyl. Even more preferably still, R² is hydrogen, methyl or acetyl. Most preferably, R² is hydrogen.

R³ is C₁-C₈alkyl, C₁-C₆haloalkyl, C₁-C₈alkoxy, C₃-C₈cycloalkyl, C₃-C₈cycloalkylC₁-C₂alkyl, wherein the cycloalkyl groups are optionally substituted with 1 to 3 groups represented by R⁶, phenyl, phenylC₁-C₂alkyl, heteroaryl, heteroarylC₁-C₂alkyl, wherein the heteroaryl is a 5-or 6-membered aromatic monocyclic ring comprising 1, 2, 3 or 4 heteroatoms individually selected from nitrogen, oxygen and sulfur, heterocyclyl, heterocyclylC₁-C₂alkyl, wherein the heterocyclyl is a 4-, 5-or 6-membered non-aromatic monocyclic ring comprising 1, 2 or 3 heteroatoms individually selected from nitrogen, oxygen and sulfur, or a 5-to 10-membered non-aromatic spirocyclic carbobi- or carbotri-cyclyl ring system optionally comprising 1, 2, or 3, heteroatoms individually selected from nitrogen, oxygen and sulfur, and optionally bonded to the rest of the molecule through a C₁-C₂alkylene linker. Preferably, R³ is C₁-C₈alkyl, C₁-C₆haloalkyl, C₁-C₈alkoxy, C₃-C₆cycloalkyl, C₃-C₆cycloalkylC₁-C₂alkyl, wherein the cycloalkyl groups are optionally substituted with 1 or 2 groups represented by R⁶, phenyl, phenylC₁-C₂alkyl, heteroaryl, heteroarylC₁-C₂alkyl, wherein the heteroaryl is a 5-or 6-membered aromatic monocyclic ring comprising 1, 2, or 3 heteroatoms individually selected from nitrogen, oxygen and sulfur, heterocyclyl, heterocyclylC₁-C₂alkyl, wherein the heterocyclyl is a 4-, 5-or 6-membered non-aromatic monocyclic ring comprising 1, 2 or 3 heteroatoms individually selected from nitrogen, oxygen and sulfur, or a 5-to 10-membered non-aromatic spirocyclic carbobi-cyclyl ring system optionally comprising 1, 2, or 3, heteroatoms individually selected from nitrogen, oxygen and sulfur, and optionally bonded to the rest of the molecule through a C₁-C₂alkylene linker. More preferably, R³ is C₁-C₈alkyl, C₁-C₆haloalkyl, C₁-C₈alkoxy, C₃-C₆cycloalkyl, C₃-C₆cycloalkylC₁-C₂alkyl, wherein the cycloalkyl groups are optionally substituted with 1 or 2 groups represented by R⁸, phenyl, heteroaryl, wherein the heteroaryl is a 5-or 6-membered aromatic monocyclic ring comprising 1, 2, or 3 heteroatoms individually selected from nitrogen, oxygen and sulfur, heterocyclyl, wherein the heterocyclyl is a 5-or 6-membered non-aromatic monocyclic ring comprising 1 or 2 heteroatoms individually selected from nitrogen, oxygen and sulfur, or a 6-to 10-membered non-aromatic spirocyclic carbobi-cyclyl ring system optionally comprising 1, or 2 heteroatoms individually selected from nitrogen, oxygen and sulfur. Even more preferably, R³ is C₁-C₈alkyl, C₁-C₆haloalkyl, C₁-C₈alkoxy, C₃-C₄cycloalkyl, C₃-C₄cycloalkylC₁-C₂alkyl, wherein the cycloalkyl groups are optionally substituted with 1 or 2 groups represented by R⁶, or a 6-to 9-membered non-aromatic spirocyclic carbobi-cyclyl ring system optionally comprising 1, or 2 heteroatoms individually selected from nitrogen, oxygen and sulfur. More preferably still, R³ is C₁-C₆alkyl, C₃-C₄cycloalkyl, wherein the cycloalkyl groups are optionally substituted with 1 or 2 groups represented by R⁶, or a 6-to 9-membered non-aromatic spirocyclic carbobi-cyclyl ring system.

In one set of embodiments, R³ is methyl, ethyl, n-propyl, isopropyl, n-butyl, t-butyl, n-pentyl, isopentyl, 2,2-dimethylpropyl, n-hexyl, cyclopropyl optionally substituted with 1 or 2 groups represented by R⁶, cyclobutyl optionally substituted with 1 or 2 groups represented by R⁶, spiro[3.3]heptan-7-yl], spiro[3.4]octan-3-yl, spiro[3.4]octan-2-yl, spiro[3.5]nonan-2-yl, or 6,6-dimethyl-7-bicyclo[3.2.0]heptanyl. Preferably, R³ is t-butyl, n-pentyl, isopentyl, 2,2-dimethylpropyl, 1-methylcyclopropyl, 2,2-dimethylcyclobutyl, or spiro[3.4]octan-3-yl. More preferably, R³ is n-pentyl, 2,2-dimethylpropyl, or spiro[3.4]octan-3-yl.

R⁴ is phenyl or heteroaryl, wherein heteroaryl is a 5-or 6-membered aromatic monocyclic ring comprising 1, 2 or 3 heteroatoms individually selected from nitrogen, oxygen and sulfur, and wherein the phenyl or heteroaryl group is optionally substituted by 1, 2 or 3 substituents, which may be the same or different, selected from R⁷. Preferably, R⁴ is phenyl or heteroaryl, wherein heteroaryl is a 5-or 6-membered aromatic monocyclic ring comprising 1, 2 or 3 heteroatoms individually selected from nitrogen, oxygen and sulfur, and wherein the phenyl or heteroaryl group is optionally substituted by 1 or 2 substituents, which may be the same or different, selected from R⁷. More preferably, R⁴ is phenyl or heteroaryl, wherein heteroaryl is a 5-or 6-membered aromatic monocyclic ring comprising 1, 2 or 3 heteroatoms individually selected from nitrogen and sulfur, and wherein the phenyl or heteroaryl group is optionally substituted by 1 or 2 substituents, which may be the same or different, selected from R⁷.

Even more preferably, R⁴ is phenyl, pyridyl, isothiazolyl, thiadiazolyl, or pyrazolyl, wherein each phenyl, pyridyl, isothiazolyl, thiadiazolyl, or pyrazolyl moiety is optionally substituted by 1 or 2 substituents, which may be the same or different, selected from R⁷.

In one set of embodiments, R⁴ is phenyl, 3-fluorophenyl, 3-chlorophenyl, 3-methylphenyl, 3-methoxyphenyl, 3,5-difluorophenyl, 3,5-dichlorophenyl, 3,5-dimethylphenyl, 3,5-dimethoxyphenyl, pyridin-4-yl, 2-fluoropyridin-4-yl, 2-chloropyridin-4-yl, 2,6-difluoropyridin-4-yl, 2,6-dichloropyridin-4-yl, pyridin-3-yl, 6-fluoropyridin-3-yl, 5-fluoropyridin-3-yl, 6-chloropyridin-3-yl, 5-chloropyridin-3-yl, isothiazol-4-yl, thiadiazol-5-yl, or 1-methylpyrazol-4-yl. More preferably still, R⁴ is 3,5-difluorophenyl, 2-fluoropyridin-4-yl, 2,6-difluoropyridin-4-yl, 5-fluoropyridin-3-yl, isothiazol-4-yl, thiadiazol-5-yl, or 1-methylpyrazol-4-yl.

R⁵ is hydrogen, halogen, or C₁-C₄alkyl. Preferably, R⁵ is hydrogen, chloro, methyl or ethyl. More preferably, R⁵ is hydrogen or methyl. More preferably still, R⁵ is hydrogen.

R⁶ is halogen, C₁-C₄alkyl, C₁-C₄alkoxy, or C₁-C₄haloalkyl. Preferably, R⁶ is chloro, fluoro, C₁-C₃alkyl, C₁-C₃alkoxy, or C₁-C₃haloalkyl. More preferably, R⁶ is chloro, fluoro, methyl, ethyl, n-propyl, isopropyl, methoxy, ethoxy, isopropoxy, difluoromethyl or trifluoromethyl. Even more preferably, R⁶ is chloro, fluoro, or methyl. Most preferably, R⁶ is methyl.

R⁷ is halogen, cyano, C₁-C₄alkyl, C₁-C₄haloalkyl, C₁-C₄alkoxy, or C₁-C₄haloalkoxy. Preferably, R⁷ is halogen, cyano, C₁-C₃alkyl, C₁-C₃haloalkyl, C₁-C₃alkoxy, or C₁-C₃haloalkoxy. More preferably, R⁷ is chloro, fluoro, cyano, C₁-C₃alkyl, C₁-C₃haloalkyl, or C₁-C₃alkoxy. Even more preferably, R⁷ is chloro, fluoro, methyl, ethyl, isopropyl, difluoromethyl, trifluoromethyl, methoxy, ethoxy or isopropoxy. In one set of embodiments R⁷ is halogen, C₁-C₃alkyl, or C₁-C₃alkoxy, preferably R⁷ is chloro, fluoro, methyl, or methoxy, and most preferably, fluoro or methyl.

In a compound of formula (I) according to the present invention, preferably:
A is N or CH;
Z is CH;
R¹ and R² are both hydrogen;
R³ is C₁-C₆alkyl, C₃-C₄cycloalkyl, wherein the cycloalkyl groups are optionally substituted with 1 or 2 groups represented by R⁶, or a 6-to 9-membered non-aromatic spirocyclic carbobi-cyclyl ring system;
R⁴ is phenyl or heteroaryl, wherein heteroaryl is a 5-or 6-membered aromatic monocyclic ring comprising 1, 2 or 3 heteroatoms individually selected from nitrogen, oxygen and sulfur, and wherein the phenyl or heteroaryl group is optionally substituted by 1 or 2 substituents, which may be the same or different, selected from R⁷;
R⁶ is methyl; and
R⁷ is halogen, C₁-C₃alkyl, or C₁-C₃alkoxy.

In another set of embodiments, A is N or CH;
Z is N or CH;
R¹ is hydrogen, cyano, or C₁-C₆alkylcarbonyl;
R² is hydrogen, C₁-C₄alkyl, or C₁-C₄alkylcarbonyl;
R³ is t-butyl, n-pentyl, isopentyl, 2,2-dimethylpropyl, 1-methylcyclopropyl, 2,2-dimethylcyclobutyl, or spiro[3.4]octan-3-yl;
R⁴ is phenyl, pyridyl, isothiazolyl, thiadiazolyl, or pyrazolyl, wherein each phenyl, pyridyl, isothiazolyl, thiadiazolyl, or pyrazolyl moiety is optionally substituted by 1 or 2 substituents, which may be the same or different, selected from R⁷; and
R⁷ is chloro, fluoro, methyl, or methoxy.

In a further set of embodiments, A is N, CH or CH;
Z is N, CH or CH;
R¹ is hydrogen, cyano, or acetyl;
R² is hydrogen, methyl, or acetyl;
R³ is t-butyl, n-pentyl, isopentyl, 2,2-dimethylpropyl, 1-methylcyclopropyl, 2,2-dimethylcyclobutyl, or spiro[3.4]octan-3-yl; and
R⁴ is phenyl, 3-fluorophenyl, 3-chlorophenyl, 3-methylphenyl, 3-methoxyphenyl, 3,5-difluorophenyl, 3,5-dichlorophenyl, 3,5-dimethylphenyl, 3,5-dimethoxyphenyl, pyridin-4-yl, 2-fluoropyridin-4-yl, 2-chloropyridin-4-yl, 2,6-difluoropyridin-4-yl, 2,6-dichloropyridin-4-yl, pyridin-3-yl, 6-fluoropyridin-3-yl, 5-fluoropyridin-3-yl, 6-chloropyridin-3-yl, 5-chloropyridin-3-yl, isothiazol-4-yl, thiadiazol-5-yl, or 1-methylpyrazol-4-yl.

In a further still set of embodiments, A is N or CH;
Z is CH;
R¹ and R² are both hydrogen;
R³ is n-pentyl, 2,2-dimethylcyclobutyl, or spiro[3.4]octan-3-yl; and
R⁴ is 3,5-difluorophenyl, 2-fluoropyridin-4-yl, 2,6-difluoropyridin-4-yl, 5-fluoropyridin-3-yl, isothiazol-4-yl, thiadiazol-5-yl, or 1-methylpyrazol-4-yl.

Compounds of the present invention can be made as shown in the following schemes, in which, unless otherwise stated, the definition of each variable is as defined above for a compound of formula (I).

The compounds of formula (I) according to the invention, wherein A, Z, R¹, R², R³ and R⁴ are as defined for formula (I), can be obtained by transformation of a compound of formula (II), wherein A, Z, R², R³ are as defined for formula (I) and R¹¹ is halogen, preferably chloro, with a compound of formula (III), wherein R¹ and R⁵ are as defined for formula (I), either by thermal heating, or with the aid of a base or under the conditions of the transition metal catalysed Buchwald-Hartwig amination. This is shown in Scheme 1 below.

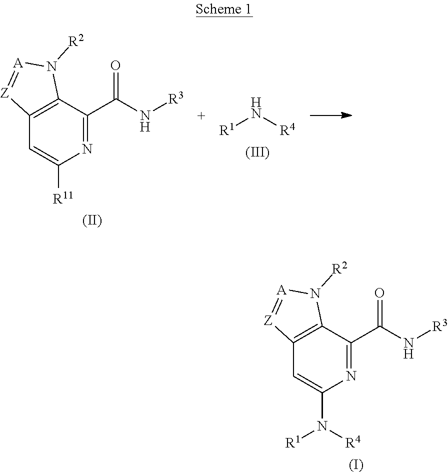

The Buchwald-Hartwig reaction is well known to those skilled in the art, and is a chemical reaction used in organic chemistry for the synthesis of carbon-nitrogen bonds via the palladium-catalyzed coupling reactions of amines with aryl and heteroaryl halides and sulphonates. Such reactions have been reported for example in for example, ACS catal., 2019, 3822-3830 and references cited therein. The reaction typically involves a palladium catalyst such Pd(OAc)₂, Pd₂(dba)₃, and ligands such diphenylphosphinobinapthyl (BINAP) and diphenylphosphinoferrocene (DPPF), and Xantphos. More modern methods of Buchwald-Hartwig couplings involve the use of palladium pre-catalysts such as BrettPhos Pd G3 (CAS[1470372-59-8]) or RuPhos Pd G3 (CAS [1445085-77-7]), use of which ensures the efficient and rapid generation of the active catalytic species. The reaction requires presence of bases such as alkaline earth metal alkoxides and hydroxides, for example potassium or sodium t-butoxides or hydroxides, alkaline earth metal carbonates such as sodium or cesium carbonates, and organic bases such as 1,8-Diazabicyclo[5.4.0]undec-7-ene (DBU). The reactions are carried out in a variety of inert solvents such as THF, water, toluene, dioxane, and DMF and/or mixtures thereof at temperatures between 20-170° C. (Buchwald, S. L. Chem. Rev., 2016. 116(19), 12564). The reaction is especially favoured in cases wherein R¹ is hydrogen.

The compounds of formula (II), wherein A, Z, R², and R³ are as defined for formula (I) and R¹¹ is halogen, preferably chloro, can be obtained by transformation of a compound of formula (IV), wherein A, Z, R² is as defined for formula (I) and R¹¹ is halogen, preferably chloro, with a compound of formula (VI), wherein R³ is as defined for formula (I), via an intermediate acid chloride or activated acylating agent as described below. This is shown in Scheme 2 below.

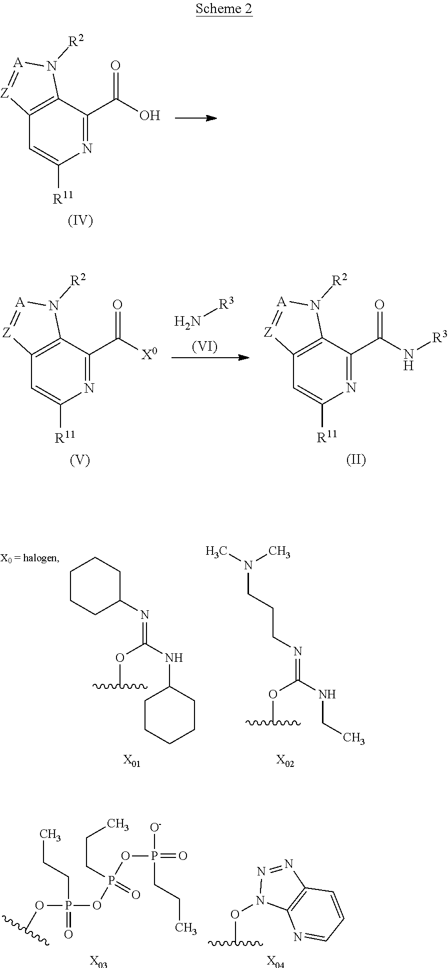

As shown in Scheme 2 compound (IV), is activated to compounds of formula (V) by methods known to those skilled in the art and described for example in Tetrahedron, 61 (46), 10827-10852, 2005. For example, compounds of formula (V) where X₀ is halogen are formed by treatment of compounds of formula (IV) with for example, oxalyl chloride or thionyl chloride in the presence of catalytic quantities of DMF in inert solvents such as methylene dichloride or THE at temperatures between 25-170° C. preferably 25-80° C. Treatment of V with compounds of formula (VI), wherein R³ is as defined in formula (I), optionally in the presence of a base, e.g. triethylamine or pyridine leads to compounds of formula II. Alternatively, compounds of formula (II) can be prepared by treatment of compounds of formula IV with dicyclohexyl carbodiimide (DCC) or 1-ethyl-3-(3-dimethylaminopropyl)carbodiimide (EDC) to give the activated species (V), wherein X₀ is X₀₁ or X₀₂, in an inert solvent, e.g. pyridine, or THE optionally in the presence of a base, e.g. triethylamine, at temperatures between room temperature and 180° C. In addition, an acid of the formula (IV) can also be activated by reaction with a coupling reagent such as propanephosphonic acid anhydride (T3P®) or O-(7-Aza-1-benzotriazolyl)-N,N,N′,N′-tetramethyluronium-hexafluorophosphate (HATU) to provide compounds of formula (V) wherein X₀ is X₀₃ and X₀₄ as described for example in Synthesis 2013, 45, 1569 and Journal Prakt. Chemie 1998, 340, 581. Subsequent reaction with an amine of the formula (VI) provides compounds of formula (II).

The compounds of formula (IV), wherein A, Z, R² is as defined for formula (I) and R¹¹ is halogen, preferably chloro, can be obtained by transformation of a compound of formula (VII), wherein A, Z, R² is as defined for formula (VII) and R¹¹ is halogen, preferably chloro, under hydrolysis conditions, preferably with the use of an inorganic acid (i.e. HCl) or with an hydroxide base (i.e. NaOH, KOH), with or without thermal heating. This is shown in Scheme 3 below.

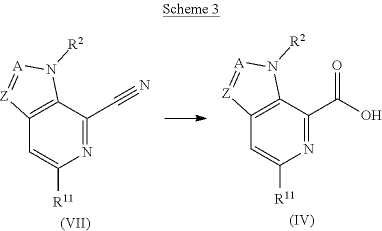

Hydrolysis of organic cyanides are well known to those skilled in the art. Examples for the hydrolysis under acidic conditions on related substrates can be found in Inorg. Chem., 2009, 48, 1753; J. Org. Chem., 1990, 55, 738; Bioorg. Med. Chem. Lett., 2007, 17, 2074. Examples for the hydrolysis under basic conditions on related substrates can be found in Tetrahedron, 2013, 69, 6799; J. Med.

Chem., 2013, 56, 1656.

The compounds of formula (VII), wherein A is heteroatom, preferably nitrogen, Z is C—R⁴, R² and R⁵ are as defined for formula (I), R¹¹ is halogen, preferably chloro, can be obtained by transformation of a compound of formula (VIII), wherein R² and R⁵ are as defined for formula (I), R¹¹ is halogen, preferably chloro, with sodium nitrite under annulation conditions. This is shown in Scheme 4 below.

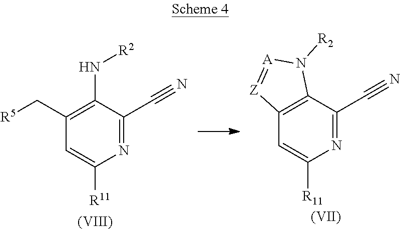

Annulation reaction of substituted anilines to substituted indazoles are known to those skilled in the art. Related examples can be found in J. Chem. Soc., Perkin Trans. 1, 1980, 2398., Bioorg. Med. Chem. Lett., 2016, 26, 5229, RSC Advances, 2016, 6, 22777, Bioorg. Med. Chem. 2007, 15, 2441.

The compounds of formula (VIII), wherein R² and R⁵ are as defined for formula (I) and R¹¹ is halogen, preferably chloro, can be obtained by transformation of a compound of formula (IX), wherein R⁵ is as defined for formula (I) and R¹¹ is halogen, preferably chloro, with a compound of formula (X), wherein R² is as defined for formula (I) and W is halogen or C₁-C₆-alkylcarbonyl, with or without the aid of a base and/or thermal heating. This is shown in Scheme 5 below.

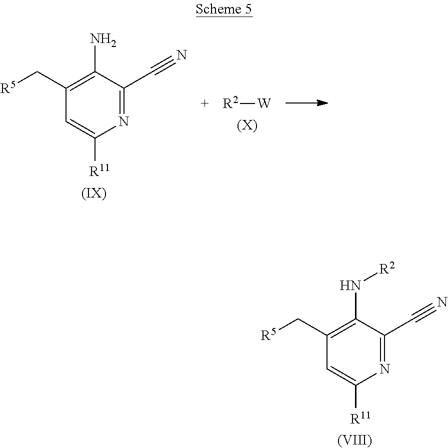

Alkylation and acylation of anilines are well known to those skilled in the art and reported examples on related substrates can be found in Chem. Eur. J., 2016, 22, 12891; Org. Proc. Res. Dev., 2014, 18, 1714; ACS Catalysis, 2017, 7, 2730;

Alternatively, compounds of formula (VIII), wherein R² and R⁵ are as defined for formula (I), R¹¹ is halogen, preferably chloro, can be obtained by transformation of a compound of formula (XII), wherein R² and R⁵ are as defined for formula (I), R¹¹ is halogen, preferably chloro, R¹³ is C₁-C₆ alkyl, under hydrolysis conditions with the aid of an hydroxide base or under acidic conditions, with or without thermal heating. This is shown in Scheme 6 below

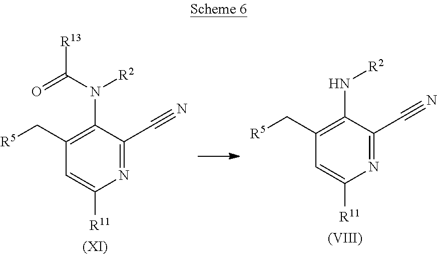

Hydrolysis of acyl anilines or aromatic imides are well known to those skilled in the art and reported examples on related substrates can be found in J. Org. Chem., 1981, 46, 3564; Synlett 2009, 11, 1741; WO 2011146287.

The compounds of formula (IX), wherein R⁵ is as defined for formula (I) and R¹¹ is halogen, preferably chloro, can be obtained by transformation of a compound of formula (XII), wherein R⁵ is as defined for formula (I), R¹¹ is halogen, preferably chloro, and R¹⁴ is halogen, preferably iodo, with ZnCN₂ either by thermal heating, or with the aid of a base or under the conditions of the transition metal catalysed coupling reaction. This is shown in Scheme 7 below

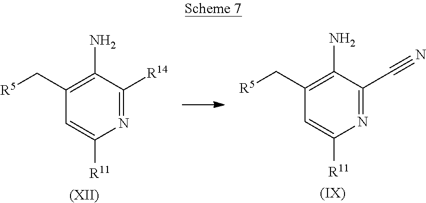

Cyanation reactions of aromatic halides are well known to those skilled in the art. Such transformation is reported to be possibly promoted by a Pd-catalyst in the presence of an appropriate ligand: Chem. Lett. 1973, 5, 471. Bull. Chem. Soc. Jpn. 1975, 48, 3298; J. Org. Chem. 2006, 71, 4021; Org. Process Res. Dev. 2008,12, 575. Tetrahedron Lett. 1999, 40, 8193; Org. Process Res. Dev. 2003, 7, 873; Org. Process Res. Dev. 2009, 13, 84; Org. Process Res. Dev. 2008, 12, 540; Tetrahedron 2006, 62, 4705. Otherwise the transformation can also be promoted by a Ni-mediator as described in J. Org. Chem. 2003, 68, 9122 or by a Cu-catalyst as described in Catal. Commun. 2009, 10, 768; Chem. Eur. J. 2007, 13, 6249; Chem. Eur. J. 2005, 11, 2483; J. Am. Chem. Soc. 2003, 125, 2829.

The compounds of formula (XII), wherein R⁵ is as defined for formula (I), R¹¹ is halogen, preferably chloro and R¹⁴ is halogen, preferably iodo, can be obtained by transformation of a compound of formula (XIII), wherein R⁵ is as defined for formula (I) and R¹¹ is halogen, preferably chloro, with an halogenating agent, preferably N-iodo succinimide, N-chloro succinimide, N-bromo succinimide or iodine. This is shown in Scheme 8 below.

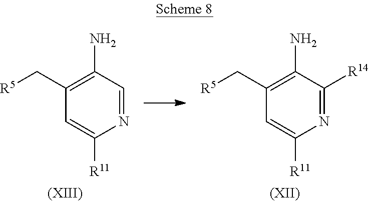

Electrophilic aromatic halogenations of anilines are well known to those skilled in the art and reported examples on related substrates can be found in EP 2014-176868, J. Med. Chem. 2013, 56, 8860; J. Org. Chem., 2015, 80,10806; Org. Lett., 2014, 16, 556.

The compounds of formula (XIII), wherein R⁵ is as defined for formula (I) and R¹¹ is halogen, preferably chloro, can be obtained by transformation of a compound of formula (XIV), wherein R⁵ is as defined for formula (I) and R¹¹ is halogen, preferably chloro, with a reducing agent, preferably hydrogen gas with or without the aid of a heterogeneous metal mediator, preferably Raney Nickel. This is shown in Scheme 9 below.

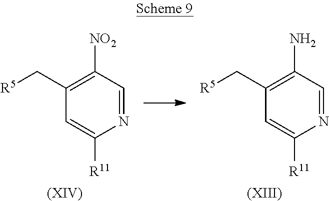

Reduction of aromatic nitro compounds to anilines can be performed under various conditions, which are well known to those skilled in the art. Standard methods are described in Comprehensive Organic Transformations; VCH: New York, 1989, pp. 411-415; Comprehensive Organic Synthesis; Pergamon Press: Oxford, 1991; Vol. 8, pp 363-379; Comprehensive Organic Functional Group Transformations; Pergamon Press: Oxford, 1995; Vol. 2, pp 737-817. Reduction of related substrates to those reported here can be found in Chemical & Pharmaceutical Bulletin, 65(1), 66-81; 2017, WO 2016095088, WO 2016141092, Synthetic Communications, 23(3), 365-72; 1993, WO 2018213211, Bioorganic & Medicinal Chemistry Letters, 18(3), 891-896; 2008; Bioorganic & Medicinal Chemistry, 19(11), 3483-3491; 2011.

Alternatively, the compounds of formula (II), wherein A, Z, R¹, R², R³ and R⁴ are as defined for formula (I), can be obtained by transformation of a compound of formula (XV), wherein A, Z, R² are as defined for formula (I), R¹¹ and R¹⁴ are halogen, preferably chloro, with a compound of formula (VI), wherein R³ is as defined for formula (I), in the presence of carbon monoxide under the conditions of transition metal catalysed aminocarbonylation. This is shown in Scheme 10 below.

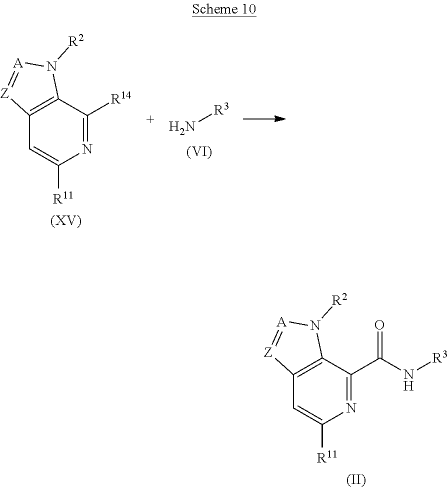

Aminocarbonylation reactions of aromatic halides are well known to those skilled in the art. The transformation, which can be promoted by a transition metal catalyst, has been pioneered by Schoenberg and Heck (J. Org. Chem. 1974, 39, 3327) and represents today a well-established access to aromatic amides (Tetrahedron 2012, 68, 9867 and references therein; Science of Synthesis: Cross-Coupling and Heck-Type Reactions; Thieme: Stuttgart, 2013. Aminocarbonylation reactions of related substrates to those reported here can be found in WO 2005121094; Eur. J. Org. Chem., 2003, 11, 2132; WO 2005082859, Bioorg. Med. Chem. Lett., 2008, 18, 5023.

The compounds of formula (XV), wherein A, Z, R² are as defined for formula (I), R¹¹ and R¹⁴ are halogen, preferably chloro, can be obtained by transformation of a compound of formula (XVI), wherein A, Z are as defined for formula (I), R¹¹ and R¹⁴ are halogen, preferably chloro, with a compound of formula (X), wherein R² is as defined for formula (I) and W is halogen or C₁-C₆-alkylcarbonyl, with or without the aid of a base and/or thermal heating. This is shown in Scheme 11 below.

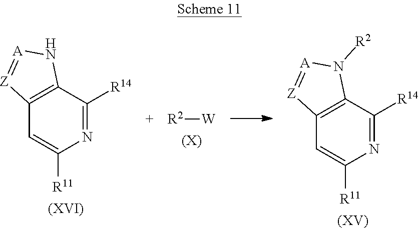

Alkylation and acylation N—H heterocycles are well known to those skilled in the art and reported examples on related substrates can be found in ACS Catalysis, 2017, 7, 7182; WO 2013037411, Heterocycles, 1989, 28, 1101; Org. Lett., 2009, 11, 1357; J. Med. Chem., 2015, 58, 9309.

The compounds of formula (XVI), wherein A and Z are C—R⁵, R⁵ and R² are as defined for formula (I), R¹¹ and R¹⁴ are halogen, preferably chloro, can be obtained by transformation of a compound of formula (XVIII), R¹¹ and R¹⁴ are halogen, preferably chloro, with a compound of formula (XVIII), wherein A, Z are C—R⁵, R⁵ is as defined for formula (I) and R¹⁵ is halogen, preferably bromine, under the conditions of Bartoli indole synthesis. This is shown in Scheme 12 below.

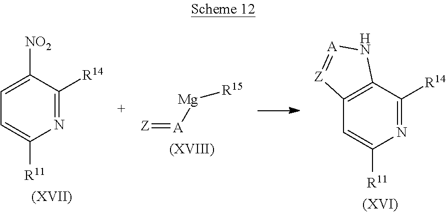

Idolization reactions are well known to those skilled in the art. In particular, the Bartoli method is well-established and largely applied to the synthesis of various indoles and related heterocycles. The field has been recently reviewed in the review article Chem. Soc. Rev., 2014, 43, 4728.

Surprisingly, it has now been found that the novel compounds of formula (I) have, for practical purposes, a very advantageous level of biological activity for protecting plants against diseases that are caused by fungi.

The compounds of formula (I) can be used in the agricultural sector and related fields of use, e.g., as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and may be used for protecting numerous cultivated plants. The compounds of formula (I) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) is applied to the plants, to parts thereof or the locus thereof.

It is also possible to use the compounds of formula (I) as fungicide. The term “fungicide” as used herein means a compound that controls, modifies, or prevents the growth of fungi. The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It is also possible to use compounds of formula (I) as dressing agents for the treatment of plant propagation material, e.g., seed, such as fruits, tubers or grains, or plant cuttings (e.g., rice), for the protection against fungal infections, as well as against phytopathogenic fungi occurring in the soil. The propagation material can be treated with a composition comprising a compound of formula (I) before planting: seed, e.g., can be dressed before being sown.

The active ingredients according to the invention can also be applied to grains (coating), either by impregnating the seeds in a liquid formulation or by coating them with a solid formulation. The composition can also be applied to the planting site when the propagation material is being planted, e.g., to the seed furrow during sowing. The invention relates also to such methods of treating plant propagation material and to the plant propagation material so treated.

Furthermore, the compounds according to present invention can be used for controlling fungi in related areas, for example in the protection of technical materials, including wood and wood related technical products, in food storage, in hygiene management.

In addition, the invention could be used to protect non-living materials from fungal attack, e.g., lumber, wall boards and paint.

The compounds of formula (I) may be, for example, effective against fungi and fungal vectors of disease as well as phytopathogenic bacteria and viruses. These fungi and fungal vectors of disease as well as phytopathogenic bacteria and viruses are for example: Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea, Coccidioides immitis, Cochliobolus spp, Colletotrichum spp. including C. musae, Cryptococcus neoformans, Diaporthe spp, Didymella spp, Drechslera spp, Elsinoe spp, Epidermophyton spp, Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi, Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp., Stereum hirsutum, Thanatephorus cucumeris, Thielaviopsis basicola, Tilletia spp, Trichoderma spp., including T. harzianum, T. pseudokoningii, T. viride, Trichophyton spp, Typhula spp, Uncinula necator, Urocystis spp, Ustilago spp, Venturia spp. including V. inaequalis, Verticillium spp, and Xanthomonas spp.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects.

Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Additionally, to date, no cross-resistance has been observed between the compounds of Formula (I) (including any one of compounds described in Table 3 (below)) and any fungicidal solutions used to control phytopathogenic fungi such as Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea, Coccidioides immitis, Cochliobolus spp, Colletotrichum spp. including C. musae, Cryptococcus neoformans, Diaporthe spp, Didymella spp, Drechslera spp, Elsinoe spp, Epidermophyton spp, Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi, Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp., Stereum hirsutum, Thanatephorus cucumeris, Thielaviopsis basicola, Tilletia spp, Trichoderma spp., including T. harzianum, T. pseudokoningii, T. viride, Trichophyton spp, Typhula spp, Uncinula necator, Urocystis spp, Ustilago spp, Venturia spp. including V. inaequalis, Verticillium spp, and Xanthomonas spp., in particular, Zymoseptoria tritici, Puccinia recondita, Puccinia striiformis, Erysiphe graminis, Uncinula necator, Sphaerotheca fuliginea, Leveillula taurica, Phakopsora pachyrhizi, Pyricularia oryzae, Alternaria solani, Alternaria alternata, Mycosphaerella fijiensis, Colletotrichum lagenarium, Didymella bryoniae, Ascochyta pisii, Verticillium dahliae, Pyrenophora teres, Cercospora beticola, Ramularia collo-cygni, Botrytis cinerea, Sclerotinia sclerotiorum, Monilinia laxa, Monographaella nivalis and Venturia inaequalis.

Indeed, fungicidal-resistant strains in any of the species as outlined above have been reported in the scientific literature, with strains resistant to one or more fungicides from at least one of the following fungicidal mode of action classes: quinone-outside-inhibitors (Qol), quinone-inside-inhibitors (Qil), succinate dehydrogenase inhibitors (SDHI) and sterol demethylation-inhibitors (DMI). Such fungicidal-resistant strains may contain:
A mutation in the mitochondrial cytochrome b gene conferring resistance to Qo inhibitors, wherein the mutation is G143A, F129L or G137R. See for example: Gisi et al., Pest Manag Sci 56, 833-841, (2000), Lucas, Pestic Outlook 14(6), 268-70 (2003), Fraaije et al., Phytopathol 95(8), 933-41 (2005), Sierotzki et al., Pest Manag Sci 63(3), 225-233 (2007), Semar et al., Journal of Plant Diseases and Protection (3), 117-119 (2007); and Pasche et al., Crop Protection 27(3-5), 427-435 (2008).
A mutation in the mitochondrial cytochrome b gene conferring resistance to Qi inhibitors, wherein the mutation is G37A/C/D/S/V. See for example: Meunier et al., Pest Manag Sci 2019; 75: 2107-2114.
A mutation in the genes encoding the SdhB,C,D subunits conferring resistance to SDHI inhibitors wherein the mutation is in the following major pathogens:
   Botrytis cinerea: B-P225H/L/TY/F, B-N2301, B-H272LY/R, C-P80H/L, C-N87S;
   Alternaria solani: B-H278R/Y, C-H134R/Q, D-D123E, D-H133R and C-H134R;
   Zymoseptoria tritici: sdhB: N225T, N225I, R265P, T268I, T268A. In sdhC: T79N, T79I, W80S, W80A, A84F, N86S, N86A, P127A, R151M/S/T/G, R151S, R151T, H152RY, V166M, T168R. In sdhD: I50F, M114V, D129G, T20P+K186R;
   Pyrenophora teres: In sdhB: S66P, N235I, H277Y. In sdhC: K49E, R64K, N75S, G79R, H134R, S135R. In sdhD: D124E, H134R, G138V, D145G;
   Ramularia collo-cygni: In sdhB: N224T, T267I. In sdhC: N87S, G91R, H146R/L, G171D, H153R;
   Phakopsora pachyrhizi: C-186F;
   Sclerotinia sclerotiorum: In sdhB: H273Y. In sdhC: G91R, H146R. In sdhD: T108K, H132R, G150R.
      Major source of information is www.frac.info, Sierotzki and Scalliet Phytopathology (2013) 103(9): 880-887 and Simões et al., J Plant Dis Prot (2018) 125: 21-2.
A mutation or combination of mutations in the CYP51 gene conferring resistance to DMI inhibitors wherein the mutations are: L50S, D134G, V136A/C, Y137F, S188N, A379G, 1381V, deletion 459-460, Y461H/S, N513K, S524T. Major source of information is www.frac.info, Cools et al., Plant Pathol (2013) 62: 36-42 and Schmitz H K et al., Pest Manag Sci (2014) 70: 378-388.

Thus, in a preferred embodiment, the compounds of Formula (I) (including any one of compounds described in Table 3 (below)), or fungicidal compositions according to the present invention comprising a compound of Formula (I), are used to control fungal strains which are resistant to one or more fungicides from any of the following fungicidal MoA classes: quinone-outside-inhibitors (Qol), quinone-inside-inhibitors (Qil), succinate dehydrogenase inhibitors (SDHI) and sterol demethylation-inhibitors (DMI).

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Pesticidal agents referred to herein using their common name are known, for example, from “The Pesticide Manual”, 15th Ed., British Crop Protection Council 2009.

The compounds of formula (I) may be used in unmodified form or, preferably, together with the adjuvants conventionally employed in the art of formulation. To this end, they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g., for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula (I) are normally used in the form of compositions and can be applied to the crop area or plant to be treated, simultaneously or in succession with further compounds. These further compounds can be, e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) may be used in the form of (fungicidal) compositions for controlling or protecting against phytopathogenic microorganisms, comprising as active ingredient at least one compound of formula (I) or of at least one preferred individual compound as above-defined, in free form or in agrochemically usable salt form, and at least one of the above-mentioned adjuvants.

The invention provides a composition, preferably a fungicidal composition, comprising at least one compound formula (I) an agriculturally acceptable carrier and optionally an adjuvant. An agricultural acceptable carrier is for example a carrier that is suitable for agricultural use. Agricultural carriers are well known in the art. Preferably, said composition may comprise at least one or more pesticidally active compounds, for example an additional fungicidal active ingredient in addition to the compound of formula (I).

The compound of formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Examples of suitable additional active ingredients also include the following: petroleum oils, 1,1-bis(4-chlorophenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxafos, benzyl benzoate, bixafen, brofenvalerate, bromocyclen, bromophos, bromopropylate, buprofezin, butocarboxim, butoxycarboxim, butylpyridaben, calcium polysulfide, camphechlor, carbanolate, carbophenothion, cymiazole, chinomethionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dinopenton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fenpyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl)methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. Iecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8, 10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B₁, trimedlure B₂, trimedlure C, trunc-call, 2-(octylthio)-ethanol, butopyronoxyl, butoxy(polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)ethane, 1,2-dichloropropane with 1,3-dichloropropene, 1-bromo-2-chloroethane, 2,2,2-trichloro-1-(3,4-dichlorophenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethylcarbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone 1. juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla), schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, and meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothio-phene 1,1-dioxide, 3-(4-chlorophenyl)-5-methylrhodanine, 5-methyl-6-thioxo-1,3,5-thiadiazinan-3-ylacetic acid, 6-isopentenylaminopurine, 2-fluoro-N-(3-methoxyphenyl)-9H-purin-6-amine, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoroacetate, thallium sulfate, warfarin, 2-(2-butoxyethoxy)ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, mercuric oxide, thiophanate-methyl, azaconazole, bitertanol, bromuconazole, cyproconazole, difenoconazole, diniconazole, epoxiconazole, fenbuconazole, fluquinconazole, flusilazole, flutriafol, furametpyr, hexaconazole, imazalil, imibenconazole, ipconazole, metconazole, myclobutanil, paclobutrazole, pefurazoate, penconazole, prothioconazole, pyrifenox, prochloraz, propiconazole, pyrisoxazole, simeconazole, tebuconazole, tetraconazole, triadimefon, triadimenol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, metalaxyl, R-metalaxyl, ofurace, oxadixyl, carbendazim, debacarb, fuberidazole, thiabendazole, chlozolinate, dichlozoline, myclozoline, procymidone, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metominostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chlorothalonil, cyflufenamid, cymoxanil, cyclobutrifluram, diclocymet, diclomezine, dicloran, diethofencarb, dimethomorph, flumorph, dithianon, ethaboxam, etridiazole, famoxadone, fenamidone, fenoxanil, ferimzone, fluazinam, fluopicolide, flusulfamide, fluxapyroxad, fenhexamid, fosetyl-aluminium, hymexazol, iprovalicarb, cyazofamid, methasulfocarb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino[1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N-(2-chloro-6-fluorophenyl)-1, 3-dimethyl-1H-pyrazol-5-amine, fluindapyr, coumethoxystrobin (jiaxiangjunzhi), Ivbenmixianan, dichlobentiazox, mandestrobin, 3-(4,4-difluoro-3,4-dihydro-3,3-dimethylisoquinolin-1-yl)quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, tebufloquin, ipflufenoquin, quinofumelin, isofetamid, N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1, 1, 3-trimethylindan-4-yl) pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, 2-(difluoromethyl)-N-((3R)-1, 1, 3-trimethylindan-4-yl) pyridine-3-carboxamide, α-(1, 1-dimethylethyl)-α-[4′-(trifluoromethoxy) [1, 1′-biphenyl]-4-yl]-5-pyrimidinemethanol, fluoxapiprolin, enoxastrobin, 4-[[6-[2-(2,4-difluorophenyl)-1,1-difluoro-2-hydroxy-3-(1,2,4-triazol-1-yl)propyl]-3-pyridyl]oxy] benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac, coumoxystrobin, zhongshengmycin, thiodiazole copper, zinc thiazole, amectotractin, iprodione, N-octyl-N′-[2-(octylamino)ethyl]ethane-1,2-diamine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl)oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl)isoquinoline, 4,4-difluoro-3,3-dimethyl-1-(7-methylpyrazolo[1,5-a]pyridin-3-yl)isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl)isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO 2017/055473, WO 2017/055469, WO 2017/093348 and WO 2017/118689); 2-[6-(4-chlorophenoxy)-2-(trifluoromethyl)-3-pyridyl]-1-(1,2,4-triazol-1-yl)propan-2-ol (this compound may be prepared from the methods described in WO 2017/029179); 2-[6-(4-bromophenoxy)-2-(trifluoromethyl)-3-pyridyl]-1-(1,2,4-triazol-1-yl)propan-2-ol (this compound may be prepared from the methods described in WO 2017/029179); 3-[2-(1-chlorocyclopropyl)-3-(2-fluorophenyl)-2-hydroxy-propyl]imidazole-4-carbonitrile (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-6-methyl-pyridine-3-carboxylate (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone (this compound may be prepared from the methods described in WO 2011/138281)N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO 2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-yl]phenyl]acetamide (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, N—[N-methoxy-C-methyl-carbonimidoyl]-4-[5-(trifluoromethyl)-1,2,4-oxadiazol-3-yl]benzamide (these compounds may be prepared from the methods described in WO 2018/202428).

The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP-357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. Nos. 5,478,855, 4,639,771 and DE-19520936.

The compounds of the invention may be used in combination with derivatives and analogues of the general class of dioxomorpholine antiparasitic agents as described in WO-9615121 and also with anthelmintic active cyclic depsipeptides such as those described in WO-9611945, WO-9319053, WO-9325543, EP-626375, EP-382173, WO-9419334, EP-382173, and EP-503538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in WO 95/19363 or WO 04/72086, particularly the compounds disclosed therein.

Other examples of such biologically active compounds that the compounds of the invention may be used in combination with include but are not restricted to the following: Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195,RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

Another aspect of invention is related to the use of a compound of formula (I) or of a preferred individual compound as above-defined, of a composition comprising at least one compound of formula (I) or at least one preferred individual compound as above-defined, or of a fungicidal or insecticidal mixture comprising at least one compound of formula (I) or at least one preferred individual compound as above-defined, in admixture with other fungicides or insecticides as described above, for controlling or preventing infestation of plants, e.g. useful plants such as crop plants, propagation material thereof, e.g. seeds, harvested crops, e.g., harvested food crops, or non-living materials by insects or by phytopathogenic microorganisms, preferably fungal organisms.

A further aspect of invention is related to a method of controlling or preventing an infestation of plants, e.g., useful plants such as crop plants, propagation material thereof, e.g. seeds, harvested crops, e.g. harvested food crops, or of non-living materials by insects or by phytopathogenic or spoilage microorganisms or organisms potentially harmful to man, especially fungal organisms, which comprises the application of a compound of formula (I) or of a preferred individual compound as above-defined as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I), or an agrochemical composition which contains at least one of said compounds, is foliar application. The frequency of application and the rate of application will depend on the risk of infestation by the corresponding pathogen or insect. However, the compounds of formula (I) can also penetrate the plant through the roots via the soil (systemic action) by drenching the locus of the plant with a liquid formulation, or by applying the compounds in solid form to the soil, e.g., in granular form (soil application). In crops of water rice such granulates can be applied to the flooded rice field. The compounds of formula (I) may also be applied to seeds (coating) by impregnating the seeds or tubers either with a liquid formulation of the fungicide or coating them with a solid formulation.

A formulation, e.g. a composition containing the compound of formula (I), and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

When the combinations of the present invention are used for treating seed, rates of 0.001 to 50 g of a compound of formula (I) per kg of seed, preferably from 0.01 to 10 g per kg of seed are generally sufficient.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g., by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g., as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) together with component (B) and (C), and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Table 1 below illustrates examples of individual compounds of formula (I) according to the invention.

TABLE 1
Individual compounds of formula
(I) according to the invention

| Cpd | | | | |
|---|---|---|---|---|
| No. | A | Z | R² | R⁴ |
| 001 | N | N | H | phenyl |
| 002 | N | N | COCH₃ | phenyl |
| 003 | N | N | CH₃ | phenyl |
| 004 | N | N | H | 3-fluorophenyl |
| 005 | N | N | COCH₃ | 3-fluorophenyl |
| 006 | N | N | CH₃ | 3-fluorophenyl |
| 007 | N | N | H | 3,5-difluorophenyl |
| 008 | N | N | COCH₃ | 3,5-difluorophenyl |
| 009 | N | N | CH₃ | 3,5-difluorophenyl |
| 010 | N | N | H | 3-chlorophenyl |
| 011 | N | N | COCH₃ | 3-chlorophenyl |
| 012 | N | N | CH₃ | 3-chlorophenyl |
| 013 | N | N | H | 3,5-dichlorophenyl |
| 014 | N | N | COCH₃ | 3,5-dichlorophenyl |
| 015 | N | N | CH₃ | 3,5-dichlorophenyl |
| 016 | N | N | H | 3-methylphenyl |
| 017 | N | N | COCH₃ | 3-methylphenyl |
| 018 | N | N | CH₃ | 3-methylphenyl |
| 019 | N | N | H | 3,5-dimethylphenyl |
| 020 | N | N | COCH₃ | 3,5-dimethylphenyl |
| 021 | N | N | CH₃ | 3,5-dimethylphenyl |
| 022 | N | N | H | 3-methoxyphenyl |
| 023 | N | N | COCH₃ | 3-methoxyphenyl |
| 024 | N | N | CH₃ | 3-methoxyphenyl |
| 025 | N | N | H | 3,5-dimethoxyphenyl |
| 026 | N | N | COCH₃ | 3,5-dimethoxyphenyl |
| 027 | N | N | CH₃ | 3,5-dimethoxyphenyl |
| 028 | N | N | H | pyridin-4-yl |
| 029 | N | N | COCH₃ | pyridin-4-yl |
| 030 | N | N | CH₃ | pyridin-4-yl |
| 031 | N | N | H | 2-fluoropyridin-4-yl |
| 032 | N | N | COCH₃ | 2-fluoropyridin-4-yl |
| 033 | N | N | CH₃ | 2-fluoropyridin-4-yl |
| 034 | N | N | H | 2,6-difluoropyridin-4-yl |
| 035 | N | N | COCH₃ | 2,6-difluoropyridin-4-yl |
| 036 | N | N | CH₃ | 2,6-difluoropyridin-4-yl |
| 037 | N | N | H | 2-chloropyridin-4-yl |
| 038 | N | N | COCH₃ | 2-chloropyridin-4-yl |
| 039 | N | N | CH₃ | 2-chloropyridin-4-yl |
| 040 | N | N | H | 2,6-dichloropyridin-4-yl |
| 041 | N | N | COCH₃ | 2,6-dichloropyridin-4-yl |
| 042 | N | N | CH₃ | 2,6-dichloropyridin-4-yl |
| 043 | N | N | H | Pyridin-3-yl |
| 044 | N | N | COCH₃ | Pyridin-3-yl |
| 045 | N | N | CH₃ | Pyridin-3-yl |
| 046 | N | N | H | 6-fluoropyridin-3-yl |
| 047 | N | N | COCH₃ | 6-fluoropyridin-3-yl |
| 048 | N | N | CH₃ | 6-fluoropyridin-3-yl |
| 049 | N | N | H | 5-fluoropyridin-3-yl |
| 050 | N | N | COCH₃ | 5-fluoropyridin-3-yl |
| 051 | N | N | CH₃ | 5-fluoropyridin-3-yl |
| 052 | N | N | H | 6-chloropyridin-3-yl |
| 053 | N | N | COCH₃ | 6-chloropyridin-3-yl |
| 054 | N | N | CH₃ | 6-chloropyridin-3-yl |
| 055 | N | N | H | 5-chloropyridin-3-yl |
| 056 | N | N | COCH₃ | 5-chloropyridin-3-yl |
| 057 | N | N | CH₃ | 5-chloropyridin-3-yl |
| 058 | N | N | H | isothiazole-4-yl |
| 059 | N | N | COCH₃ | isothiazole-4-yl |
| 060 | N | N | CH₃ | isothiazole-4-yl |
| 061 | N | CH | H | phenyl |
| 062 | N | CH | COCH₃ | phenyl |
| 063 | N | CH | CH₃ | phenyl |
| 064 | N | CH | H | 3-fluorophenyl |
| 065 | N | CH | COCH₃ | 3-fluorophenyl |
| 066 | N | CH | CH₃ | 3-fluorophenyl |
| 067 | N | CH | H | 3,5-difluorophenyl |
| 068 | N | CH | COCH₃ | 3,5-difluorophenyl |
| 069 | N | CH | CH₃ | 3,5-difluorophenyl |
| 070 | N | CH | H | 3-chlorophenyl |
| 071 | N | CH | COCH₃ | 3-chlorophenyl |
| 072 | N | CH | CH₃ | 3-chlorophenyl |
| 073 | N | CH | H | 3,5-dichlorophenyl |
| 074 | N | CH | COCH₃ | 3,5-dichlorophenyl |
| 075 | N | CH | CH₃ | 3,5-dichlorophenyl |
| 076 | N | CH | H | 3-methylphenyl |
| 077 | N | CH | COCH₃ | 3-methylphenyl |
| 078 | N | CH | CH₃ | 3-methylphenyl |
| 079 | N | CH | H | 3,5-dimethylphenyl |
| 080 | N | CH | COCH₃ | 3,5-dimethylphenyl |
| 081 | N | CH | CH₃ | 3,5-dimethylphenyl |
| 082 | N | CH | H | 3-methoxyphenyl |
| 083 | N | CH | COCH₃ | 3-methoxyphenyl |
| 084 | N | CH | CH₃ | 3-methoxyphenyl |
| 085 | N | CH | H | 3,5-dimethoxyphenyl |
| 086 | N | CH | COCH₃ | 3,5-dimethoxyphenyl |
| 087 | N | CH | CH₃ | 3,5-dimethoxyphenyl |
| 088 | N | CH | H | pyridin-4-yl |
| 089 | N | CH | COCH₃ | pyridin-4-yl |
| 090 | N | CH | CH₃ | pyridin-4-yl |
| 091 | N | CH | H | 2-fluoropyridin-4-yl |
| 092 | N | CH | COCH₃ | 2-fluoropyridin-4-yl |
| 093 | N | CH | CH₃ | 2-fluoropyridin-4-yl |
| 094 | N | CH | H | 2,6-difluoropyridin-4-yl |
| 095 | N | CH | COCH₃ | 2,6-difluoropyridin-4-yl |
| 096 | N | CH | CH₃ | 2,6-difluoropyridin-4-yl |
| 097 | N | CH | H | 2-chloropyridin-4-yl |
| 098 | N | CH | COCH₃ | 2-chloropyridin-4-yl |
| 099 | N | CH | CH₃ | 2-chloropyridin-4-yl |
| 100 | N | CH | H | 2,6-dichloropyridin-4-yl |
| 101 | N | CH | COCH₃ | 2,6-dichloropyridin-4-yl |
| 102 | N | CH | CH₃ | 2,6-dichloropyridin-4-yl |
| 103 | N | CH | H | pyridin-3-yl |
| 104 | N | CH | COCH₃ | pyridin-3-yl |
| 105 | N | CH | CH₃ | pyridin-3-yl |
| 106 | N | CH | H | 6-fluoropyridin-3-yl |
| 107 | N | CH | COCH₃ | 6-fluoropyridin-3-yl |
| 108 | N | CH | CH₃ | 6-fluoropyridin-3-yl |
| 109 | N | CH | H | 5-fluoropyridin-3-yl |
| 110 | N | CH | COCH₃ | 5-fluoropyridin-3-yl |
| 111 | N | CH | CH₃ | 5-fluoropyridin-3-yl |
| 112 | N | CH | H | 6-chloropyridin-3-yl |
| 113 | N | CH | COCH₃ | 6-chloropyridin-3-yl |
| 114 | N | CH | CH₃ | 6-chloropyridin-3-yl |
| 115 | N | CH | H | 5-chloropyridin-3-yl |
| 116 | N | CH | COCH₃ | 5-chloropyridin-3-yl |
| 117 | N | CH | CH₃ | 5-chloropyridin-3-yl |
| 118 | N | CH | H | isothiazole-4-yl |
| 119 | N | CH | COCH₃ | isothiazole-4-yl |
| 120 | N | CH | CH₃ | isothiazole-4-yl |
| 121 | N | CCH₃ | H | phenyl |
| 122 | N | CCH₃ | COCH₃ | phenyl |
| 123 | N | CCH₃ | CH₃ | phenyl |
| 124 | N | CCH₃ | H | 3-fluorophenyl |
| 125 | N | CCH₃ | COCH₃ | 3-fluorophenyl |
| 126 | N | CCH₃ | CH₃ | 3-fluorophenyl |
| 127 | N | CCH₃ | H | 3,5-difluorophenyl |
| 128 | N | CCH₃ | COCH₃ | 3,5-difluorophenyl |
| 129 | N | CCH₃ | CH₃ | 3,5-difluorophenyl |
| 130 | N | CCH₃ | H | 3-chlorophenyl |
| 131 | N | CCH₃ | COCH₃ | 3-chlorophenyl |
| 132 | N | CCH₃ | CH₃ | 3-chlorophenyl |
| 133 | N | CCH₃ | H | 3,5-dichlorophenyl |
| 134 | N | CCH₃ | COCH₃ | 3,5-dichlorophenyl |
| 135 | N | CCH₃ | CH₃ | 3,5-dichlorophenyl |
| 136 | N | CCH₃ | H | 3-methylphenyl |
| 137 | N | CCH₃ | COCH₃ | 3-methylphenyl |
| 138 | N | CCH₃ | CH₃ | 3-methylphenyl |
| 139 | N | CCH₃ | H | 3,5-dimethylphenyl |
| 140 | N | CCH₃ | COCH₃ | 3,5-dimethylphenyl |
| 141 | N | CCH₃ | CH₃ | 3,5-dimethylphenyl |
| 142 | N | CCH₃ | H | 3-methoxyphenyl |
| 143 | N | CCH₃ | COCH₃ | 3-methoxyphenyl |
| 144 | N | CCH₃ | CH₃ | 3-methoxyphenyl |
| 145 | N | CCH₃ | H | 3,5-dimethoxyphenyl |
| 146 | N | CCH₃ | COCH₃ | 3,5-dimethoxyphenyl |
| 147 | N | CCH₃ | CH₃ | 3,5-dimethoxyphenyl |
| 148 | N | CCH₃ | H | pyridin-4-yl |
| 149 | N | CCH₃ | COCH₃ | pyridin-4-yl |
| 150 | N | CCH₃ | CH₃ | pyridin-4-yl |
| 151 | N | CCH₃ | H | 2-fluoropyridin-4-yl |
| 152 | N | CCH₃ | COCH₃ | 2-fluoropyridin-4-yl |
| 153 | N | CCH₃ | CH₃ | 2-fluoropyridin-4-yl |
| 154 | N | CCH₃ | H | 2,6-difluoropyridin-4-yl |
| 155 | N | CCH₃ | COCH₃ | 2,6-difluoropyridin-4-yl |
| 156 | N | CCH₃ | CH₃ | 2,6-difluoropyridin-4-yl |
| 157 | N | CCH₃ | H | 2-chloropyridin-4-yl |
| 158 | N | CCH₃ | COCH₃ | 2-chloropyridin-4-yl |
| 159 | N | CCH₃ | CH₃ | 2-chloropyridin-4-yl |
| 160 | N | CCH₃ | H | 2,6-dichloropyridin-4-yl |
| 161 | N | CCH₃ | COCH₃ | 2,6-dichloropyridin-4-yl |
| 162 | N | CCH₃ | CH₃ | 2,6-dichloropyridin-4-yl |
| 163 | N | CCH₃ | H | pyridin-3-yl |
| 164 | N | CCH₃ | COCH₃ | pyridin-3-yl |
| 165 | N | CCH₃ | CH₃ | pyridin-3-yl |
| 166 | N | CCH₃ | H | 6-fluoropyridin-3-yl |
| 167 | N | CCH₃ | COCH₃ | 6-fluoropyridin-3-yl |
| 168 | N | CCH₃ | CH₃ | 6-fluoropyridin-3-yl |
| 169 | N | CCH₃ | H | 5-fluoropyridin-3-yl |
| 170 | N | CCH₃ | COCH₃ | 5-fluoropyridin-3-yl |
| 171 | N | CCH₃ | CH₃ | 5-fluoropyridin-3-yl |
| 172 | N | CCH₃ | H | 6-chloropyridin-3-yl |
| 173 | N | CCH₃ | COCH₃ | 6-chloropyridin-3-yl |
| 174 | N | CCH₃ | CH₃ | 6-chloropyridin-3-yl |
| 175 | N | CCH₃ | H | 5-chloropyridin-3-yl |
| 176 | N | CCH₃ | COCH₃ | 5-chloropyridin-3-yl |
| 177 | N | CCH₃ | CH₃ | 5-chloropyridin-3-yl |
| 178 | N | CCH₃ | H | isothiazole-4-yl |
| 179 | N | CCH₃ | COCH₃ | isothiazole-4-yl |
| 180 | N | CCH₃ | CH₃ | isothiazole-4-yl |
| 181 | CH | N | H | phenyl |
| 182 | CH | N | COCH₃ | phenyl |
| 183 | CH | N | CH₃ | phenyl |
| 184 | CH | N | H | 3-fluorophenyl |
| 185 | CH | N | COCH₃ | 3-fluorophenyl |
| 186 | CH | N | CH₃ | 3-fluorophenyl |
| 187 | CH | N | H | 3,5-difluorophenyl |
| 188 | CH | N | COCH₃ | 3,5-difluorophenyl |
| 189 | CH | N | CH₃ | 3,5-difluorophenyl |
| 190 | CH | N | H | 3-chlorophenyl |
| 191 | CH | N | COCH₃ | 3-chlorophenyl |
| 192 | CH | N | CH₃ | 3-chlorophenyl |
| 193 | CH | N | H | 3,5-dichlorophenyl |
| 194 | CH | N | COCH₃ | 3,5-dichlorophenyl |
| 195 | CH | N | CH₃ | 3,5-dichlorophenyl |
| 196 | CH | N | H | 3-methylphenyl |
| 197 | CH | N | COCH₃ | 3-methylphenyl |
| 198 | CH | N | CH₃ | 3-methylphenyl |
| 199 | CH | N | H | 3,5-dimethylphenyl |
| 200 | CH | N | COCH₃ | 3,5-dimethylphenyl |
| 201 | CH | N | CH₃ | 3,5-dimethylphenyl |
| 202 | CH | N | H | 3-methoxyphenyl |
| 203 | CH | N | COCH₃ | 3-methoxyphenyl |
| 204 | CH | N | CH₃ | 3-methoxyphenyl |
| 205 | CH | N | H | 3,5-dimethoxyphenyl |
| 206 | CH | N | COCH₃ | 3,5-dimethoxyphenyl |
| 207 | CH | N | CH₃ | 3,5-dimethoxyphenyl |
| 208 | CH | N | H | pyridin-4-yl |
| 209 | CH | N | COCH₃ | pyridin-4-yl |
| 210 | CH | N | CH₃ | pyridin-4-yl |
| 211 | CH | N | H | 2-fluoropyridin-4-yl |
| 212 | CH | N | COCH₃ | 2-fluoropyridin-4-yl |
| 213 | CH | N | CH₃ | 2-fluoropyridin-4-yl |
| 214 | CH | N | H | 2,6-difluoropyridin-4-yl |
| 215 | CH | N | COCH₃ | 2,6-difluoropyridin-4-yl |
| 216 | CH | N | CH₃ | 2,6-difluoropyridin-4-yl |
| 217 | CH | N | H | 2-chloropyridin-4-yl |
| 218 | CH | N | COCH₃ | 2-chloropyridin-4-yl |
| 219 | CH | N | CH₃ | 2-chloropyridin-4-yl |
| 220 | CH | N | H | 2,6-dichloropyridin-4-yl |
| 221 | CH | N | COCH₃ | 2,6-dichloropyridin-4-yl |
| 222 | CH | N | CH₃ | 2,6-dichloropyridin-4-yl |
| 223 | CH | N | H | pyridin-3-yl |
| 224 | CH | N | COCH₃ | pyridin-3-yl |
| 225 | CH | N | CH₃ | pyridin-3-yl |
| 226 | CH | N | H | 6-fluoropyridin-3-yl |
| 227 | CH | N | COCH₃ | 6-fluoropyridin-3-yl |
| 228 | CH | N | CH₃ | 6-fluoropyridin-3-yl |
| 229 | CH | N | H | 5-fluoropyridin-3-yl |
| 230 | CH | N | COCH₃ | 5-fluoropyridin-3-yl |
| 231 | CH | N | CH₃ | 5-fluoropyridin-3-yl |
| 232 | CH | N | H | 6-chloropyridin-3-yl |
| 233 | CH | N | COCH₃ | 6-chloropyridin-3-yl |
| 234 | CH | N | CH₃ | 6-chloropyridin-3-yl |
| 235 | CH | N | H | 5-chloropyridin-3-yl |
| 236 | CH | N | COCH₃ | 5-chloropyridin-3-yl |
| 237 | CH | N | CH₃ | 5-chloropyridin-3-yl |
| 238 | CH | N | H | isothiazole-4-yl |
| 239 | CH | N | COCH₃ | isothiazole-4-yl |
| 240 | CH | N | CH₃ | isothiazole-4-yl |
| 241 | CH | CH | H | phenyl |
| 242 | CH | CH | COCH₃ | phenyl |
| 243 | CH | CH | CH₃ | phenyl |
| 244 | CH | CH | H | 3-fluorophenyl |
| 245 | CH | CH | COCH₃ | 3-fluorophenyl |
| 246 | CH | CH | CH₃ | 3-fluorophenyl |
| 247 | CH | CH | H | 3,5-difluorophenyl |
| 248 | CH | CH | COCH₃ | 3,5-difluorophenyl |
| 249 | CH | CH | CH₃ | 3,5-difluorophenyl |
| 250 | CH | CH | H | 3-chlorophenyl |
| 251 | CH | CH | COCH₃ | 3-chlorophenyl |
| 252 | CH | CH | CH₃ | 3-chlorophenyl |
| 253 | CH | CH | H | 3,5-dichlorophenyl |
| 254 | CH | CH | COCH₃ | 3,5-dichlorophenyl |
| 255 | CH | CH | CH₃ | 3,5-dichlorophenyl |
| 256 | CH | CH | H | 3-methylphenyl |
| 257 | CH | CH | COCH₃ | 3-methylphenyl |
| 258 | CH | CH | CH₃ | 3-methylphenyl |
| 259 | CH | CH | H | 3,5-dimethylphenyl |
| 260 | CH | CH | COCH₃ | 3,5-dimethylphenyl |
| 261 | CH | CH | CH₃ | 3,5-dimethylphenyl |
| 262 | CH | CH | H | 3-methoxyphenyl |
| 263 | CH | CH | COCH₃ | 3-methoxyphenyl |
| 264 | CH | CH | CH₃ | 3-methoxyphenyl |
| 265 | CH | CH | H | 3,5-dimethoxyphenyl |
| 266 | CH | CH | COCH₃ | 3,5-dimethoxyphenyl |
| 267 | CH | CH | CH₃ | 3,5-dimethoxyphenyl |
| 268 | CH | CH | H | pyridin-4-yl |
| 269 | CH | CH | COCH₃ | pyridin-4-yl |
| 270 | CH | CH | CH₃ | pyridin-4-yl |
| 271 | CH | CH | H | 2-fluoropyridin-4-yl |
| 272 | CH | CH | COCH₃ | 2-fluoropyridin-4-yl |
| 273 | CH | CH | CH₃ | 2-fluoropyridin-4-yl |
| 274 | CH | CH | H | 2,6-difluoropyridin-4-yl |
| 275 | CH | CH | COCH₃ | 2,6-difluoropyridin-4-yl |
| 276 | CH | CH | CH₃ | 2,6-difluoropyridin-4-yl |
| 277 | CH | CH | H | 2-chloropyridin-4-yl |
| 278 | CH | CH | COCH₃ | 2-chloropyridin-4-y1 |
| 279 | CH | CH | CH₃ | 2-chloropyridin-4-y1 |
| 280 | CH | CH | H | 2,6-dichloropyridin-4-yl |
| 281 | CH | CH | COCH₃ | 2,6-dichloropyridin-4-yl |
| 282 | CH | CH | CH₃ | 2,6-dichloropyridin-4-yl |
| 283 | CH | CH | H | pyridin-3-yl |
| 284 | CH | CH | COCH₃ | pyridin-3-yl |
| 285 | CH | CH | CH₃ | pyridin-3-yl |
| 286 | CH | CH | H | 6-fluoropyridin-3-yl |
| 287 | CH | CH | COCH₃ | 6-fluoropyridin-3-yl |
| 288 | CH | CH | CH₃ | 6-fluoropyridin-3-yl |
| 289 | CH | CH | H | 5-fluoropyridin-3-yl |
| 290 | CH | CH | COCH₃ | 5-fluoropyridin-3-yl |
| 291 | CH | CH | CH₃ | 5-fluoropyridin-3-yl |
| 292 | CH | CH | H | 6-chloropyridin-3-yl |
| 293 | CH | CH | COCH₃ | 6-chloropyridin-3-yl |
| 294 | CH | CH | CH₃ | 6-chloropyridin-3-yl |
| 295 | CH | CH | H | 5-chloropyridin-3-yl |
| 296 | CH | CH | COCH₃ | 5-chloropyridin-3-yl |
| 297 | CH | CH | CH₃ | 5-chloropyridin-3-yl |
| 298 | CH | CH | H | isothiazole-4-yl |
| 299 | CH | CH | COCH₃ | isothiazole-4-yl |
| 300 | CH | CH | CH₃ | isothiazole-4-yl |
| 301 | CH | CCH₃ | H | phenyl |
| 302 | CH | CCH₃ | COCH₃ | phenyl |
| 303 | CH | CCH₃ | CH₃ | phenyl |
| 304 | CH | CCH₃ | H | 3-fluorophenyl |
| 305 | CH | CCH₃ | COCH₃ | 3-fluorophenyl |
| 306 | CH | CCH₃ | CH₃ | 3-fluorophenyl |
| 307 | CH | CCH₃ | H | 3,5-difluorophenyl |
| 308 | CH | CCH₃ | COCH₃ | 3,5-difluorophenyl |
| 309 | CH | CCH₃ | CH₃ | 3,5-difluorophenyl |
| 310 | CH | CCH₃ | H | 3-chlorophenyl |
| 311 | CH | CCH₃ | COCH₃ | 3-chlorophenyl |
| 312 | CH | CCH₃ | CH₃ | 3-chlorophenyl |
| 313 | CH | CCH₃ | H | 3,5-dichlorophenyl |
| 314 | CH | CCH₃ | COCH₃ | 3,5-dichlorophenyl |
| 315 | CH | CCH₃ | CH₃ | 3,5-dichlorophenyl |
| 316 | CH | CCH₃ | H | 3-methylphenyl |
| 317 | CH | CCH₃ | COCH₃ | 3-methylphenyl |
| 318 | CH | CCH₃ | CH₃ | 3-methylphenyl |
| 319 | CH | CCH₃ | H | 3,5-dimethylphenyl |
| 320 | CH | CCH₃ | COCH₃ | 3,5-dimethylphenyl |
| 321 | CH | CCH₃ | CH₃ | 3,5-dimethylphenyl |
| 322 | CH | CCH₃ | H | 3-methoxyphenyl |
| 323 | CH | CCH₃ | COCH₃ | 3-methoxyphenyl |
| 324 | CH | CCH₃ | CH₃ | 3-methoxyphenyl |
| 325 | CH | CCH₃ | H | 3,5-dimethoxyphenyl |
| 326 | CH | CCH₃ | COCH₃ | 3,5-dimethoxyphenyl |
| 327 | CH | CCH₃ | CH₃ | 3,5-dimethoxyphenyl |
| 328 | CH | CCH₃ | H | pyridin-4-yl |
| 329 | CH | CCH₃ | COCH₃ | pyridin-4-yl |
| 330 | CH | CCH₃ | CH₃ | pyridin-4-yl |
| 331 | CH | CCH₃ | H | 2-fluoropyridin-4-yl |
| 332 | CH | CCH₃ | COCH₃ | 2-fluoropyridin-4-yl |
| 333 | CH | CCH₃ | CH₃ | 2-fluoropyridin-4-yl |
| 334 | CH | CCH₃ | H | 2,6-difluoropyridin-4-yl |
| 335 | CH | CCH₃ | COCH₃ | 2,6-difluoropyridin-4-yl |
| 336 | CH | CCH₃ | CH₃ | 2,6-difluoropyridin-4-yl |
| 337 | CH | CCH₃ | H | 2-chloropyridin-4-yl |
| 338 | CH | CCH₃ | COCH₃ | 2-chloropyridin-4-yl |
| 339 | CH | CCH₃ | CH₃ | 2-chloropyridin-4-yl |
| 340 | CH | CCH₃ | H | 2,6-dichloropyridin-4-yl |
| 341 | CH | CCH₃ | COCH₃ | 2,6-dichloropyridin-4-yl |
| 342 | CH | CCH₃ | CH₃ | 2,6-dichloropyridin-4-yl |
| 343 | CH | CCH₃ | H | pyridin-3-yl |
| 344 | CH | CCH₃ | COCH₃ | pyridin-3-yl |
| 345 | CH | CCH₃ | CH₃ | pyridin-3-yl |
| 346 | CH | CCH₃ | H | 6-fluoropyridin-3-yl |
| 347 | CH | CCH₃ | COCH₃ | 6-fluoropyridin-3-yl |
| 348 | CH | CCH₃ | CH₃ | 6-fluoropyridin-3-yl |
| 349 | CH | CCH₃ | H | 5-fluoropyridin-3-yl |
| 350 | CH | CCH₃ | COCH₃ | 5-fluoropyridin-3-yl |
| 351 | CH | CCH₃ | CH₃ | 5-fluoropyridin-3-yl |
| 352 | CH | CCH₃ | H | 6-chloropyridin-3-yl |
| 353 | CH | CCH₃ | COCH₃ | 6-chloropyridin-3-yl |
| 354 | CH | CCH₃ | CH₃ | 6-chloropyridin-3-yl |
| 355 | CH | CCH₃ | H | 5-chloropyridin-3-yl |
| 356 | CH | CCH₃ | COCH₃ | 5-chloropyridin-3-yl |
| 357 | CH | CCH₃ | CH₃ | 5-chloropyridin-3-yl |
| 358 | CH | CCH₃ | H | isothiazole-4-yl |
| 359 | CH | CCH₃ | COCH₃ | isothiazole-4-yl |
| 360 | CH | CCH₃ | CH₃ | isothiazole-4-yl |
| 361 | CCH₃ | N | H | phenyl |
| 362 | CCH₃ | N | COCH₃ | phenyl |
| 363 | CCH₃ | N | CH₃ | phenyl |
| 364 | CCH₃ | N | H | 3-fluorophenyl |
| 365 | CCH₃ | N | COCH₃ | 3-fluorophenyl |
| 366 | CCH₃ | N | CH₃ | 3-fluorophenyl |
| 367 | CCH₃ | N | H | 3,5-difluorophenyl |
| 368 | CCH₃ | N | COCH₃ | 3,5-difluorophenyl |
| 369 | CCH₃ | N | CH₃ | 3,5-difluorophenyl |
| 370 | CCH₃ | N | H | 3-chlorophenyl |
| 371 | CCH₃ | N | COCH₃ | 3-chlorophenyl |
| 372 | CCH₃ | N | CH₃ | 3-chlorophenyl |
| 373 | CCH₃ | N | H | 3,5-dichlorophenyl |
| 374 | CCH₃ | N | COCH₃ | 3,5-dichlorophenyl |
| 375 | CCH₃ | N | CH₃ | 3,5-dichlorophenyl |
| 376 | CCH₃ | N | H | 3-methylphenyl |
| 377 | CCH₃ | N | COCH₃ | 3-methylphenyl |
| 378 | CCH₃ | N | CH₃ | 3-methylphenyl |
| 379 | CCH₃ | N | H | 3,5-dimethylphenyl |
| 380 | CCH₃ | N | COCH₃ | 3,5-dimethylphenyl |
| 381 | CCH₃ | N | CH₃ | 3,5-dimethylphenyl |
| 382 | CCH₃ | N | H | 3-methoxyphenyl |
| 383 | CCH₃ | N | COCH₃ | 3-methoxyphenyl |
| 384 | CCH₃ | N | CH₃ | 3-methoxyphenyl |
| 385 | CCH₃ | N | H | 3,5-dimethoxyphenyl |
| 386 | CCH₃ | N | COCH₃ | 3,5-dimethoxyphenyl |
| 387 | CCH₃ | N | CH₃ | 3,5-dimethoxyphenyl |
| 388 | CCH₃ | N | H | pyridin-4-yl |
| 389 | CCH₃ | N | COCH₃ | pyridin-4-yl |
| 390 | CCH₃ | N | CH₃ | pyridin-4-yl |
| 391 | CCH₃ | N | H | 2-fluoropyridin-4-yl |
| 392 | CCH₃ | N | COCH₃ | 2-fluoropyridin-4-yl |
| 393 | CCH₃ | N | CH₃ | 2-fluoropyridin-4-yl |
| 394 | CCH₃ | N | H | 2,6-difluoropyridin-4-yl |
| 395 | CCH₃ | N | COCH₃ | 2,6-difluoropyridin-4-yl |
| 396 | CCH₃ | N | CH₃ | 2,6-difluoropyridin-4-yl |
| 397 | CCH₃ | N | H | 2-chloropyridin-4-yl |
| 398 | CCH₃ | N | COCH₃ | 2-chloropyridin-4-yl |
| 399 | CCH₃ | N | CH₃ | 2-chloropyridin-4-yl |
| 400 | CCH₃ | N | H | 2,6-dichloropyridin-4-yl |
| 401 | CCH₃ | N | COCH₃ | 2,6-dichloropyridin-4-yl |
| 402 | CCH₃ | N | CH₃ | 2,6-dichloropyridin-4-yl |
| 403 | CCH₃ | N | H | pyridin-3-yl |
| 404 | CCH₃ | N | COCH₃ | pyridin-3-yl |
| 405 | CCH₃ | N | CH₃ | pyridin-3-yl |
| 406 | CCH₃ | N | H | 6-fluoropyridin-3-yl |
| 407 | CCH₃ | N | COCH₃ | 6-fluoropyridin-3-yl |
| 408 | CCH₃ | N | CH₃ | 6-fluoropyridin-3-yl |
| 409 | CCH₃ | N | H | 5-fluoropyridin-3-yl |
| 410 | CCH₃ | N | COCH₃ | 5-fluoropyridin-3-yl |
| 411 | CCH₃ | N | CH₃ | 5-fluoropyridin-3-yl |
| 412 | CCH₃ | N | H | 6-chloropyridin-3-yl |
| 413 | CCH₃ | N | COCH₃ | 6-chloropyridin-3-yl |
| 414 | CCH₃ | N | CH₃ | 6-chloropyridin-3-yl |
| 415 | CCH₃ | N | H | 5-chloropyridin-3-yl |
| 416 | CCH₃ | N | COCH₃ | 5-chloropyridin-3-yl |
| 417 | CCH₃ | N | CH₃ | 5-chloropyridin-3-yl |
| 418 | CCH₃ | N | H | isothiazole-4-yl |
| 419 | CCH₃ | N | COCH₃ | isothiazole-4-yl |
| 420 | CCH₃ | N | CH₃ | isothiazole-4-yl |
| 421 | CCH₃ | CH | H | phenyl |
| 422 | CCH₃ | CH | COCH₃ | phenyl |
| 423 | CCH₃ | CH | CH₃ | phenyl |
| 424 | CCH₃ | CH | H | 3-fluorophenyl |
| 425 | CCH₃ | CH | COCH₃ | 3-fluorophenyl |
| 426 | CCH₃ | CH | CH₃ | 3-fluorophenyl |
| 427 | CCH₃ | CH | H | 3,5-difluorophenyl |
| 428 | CCH₃ | CH | COCH₃ | 3,5-difluorophenyl |
| 429 | CCH₃ | CH | CH₃ | 3,5-difluorophenyl |
| 430 | CCH₃ | CH | H | 3-chlorophenyl |
| 431 | CCH₃ | CH | COCH₃ | 3-chlorophenyl |
| 432 | CCH₃ | CH | CH₃ | 3-chlorophenyl |
| 433 | CCH₃ | CH | H | 3,5-dichlorophenyl |
| 434 | CCH₃ | CH | COCH₃ | 3,5-dichlorophenyl |
| 435 | CCH₃ | CH | CH₃ | 3,5-dichlorophenyl |
| 436 | CCH₃ | CH | H | 3-methylphenyl |
| 437 | CCH₃ | CH | COCH₃ | 3-methylphenyl |
| 438 | CCH₃ | CH | CH₃ | 3-methylphenyl |
| 439 | CCH₃ | CH | H | 3,5-dimethylphenyl |
| 440 | CCH₃ | CH | COCH₃ | 3,5-dimethylphenyl |
| 441 | CCH₃ | CH | CH₃ | 3,5-dimethylphenyl |
| 442 | CCH₃ | CH | H | 3-methoxyphenyl |
| 443 | CCH₃ | CH | COCH₃ | 3-methoxyphenyl |
| 444 | CCH₃ | CH | CH₃ | 3-methoxyphenyl |
| 445 | CCH₃ | CH | H | 3,5-dimethoxyphenyl |
| 446 | CCH₃ | CH | COCH₃ | 3,5-dimethoxyphenyl |
| 447 | CCH₃ | CH | CH₃ | 3,5-dimethoxyphenyl |
| 448 | CCH₃ | CH | H | pyridin-4-yl |
| 449 | CCH₃ | CH | COCH₃ | pyridin-4-yl |
| 450 | CCH₃ | CH | CH₃ | pyridin-4-yl |
| 451 | CCH₃ | CH | H | 2-fluoropyridin-4-yl |
| 452 | CCH₃ | CH | COCH₃ | 2-fluoropyridin-4-yl |
| 453 | CCH₃ | CH | CH₃ | 2-fluoropyridin-4-yl |
| 454 | CCH₃ | CH | H | 2,6-difluoropyridin-4-yl |
| 455 | CCH₃ | CH | COCH₃ | 2,6-difluoropyridin-4-yl |
| 456 | CCH₃ | CH | CH₃ | 2,6-difluoropyridin-4-yl |
| 457 | CCH₃ | CH | H | 2-chloropyridin-4-yl |
| 458 | CCH₃ | CH | COCH₃ | 2-chloropyridin-4-yl |
| 459 | CCH₃ | CH | CH₃ | 2-chloropyridin-4-yl |
| 460 | CCH₃ | CH | H | 2,6-dichloropyridin-4-yl |
| 461 | CCH₃ | CH | COCH₃ | 2,6-dichloropyridin-4-yl |
| 462 | CCH₃ | CH | CH₃ | 2,6-dichloropyridin-4-yl |
| 463 | CCH₃ | CH | H | pyridin-3-yl |
| 464 | CCH₃ | CH | COCH₃ | pyridin-3-yl |
| 465 | CCH₃ | CH | CH₃ | pyridin-3-yl |
| 466 | CCH₃ | CH | H | 6-fluoropyridin-3-yl |
| 467 | CCH₃ | CH | COCH₃ | 6-fluoropyridin-3-yl |
| 468 | CCH₃ | CH | CH₃ | 6-fluoropyridin-3-yl |
| 469 | CCH₃ | CH | H | 5-fluoropyridin-3-yl |
| 470 | CCH₃ | CH | COCH₃ | 5-fluoropyridin-3-yl |
| 471 | CCH₃ | CH | CH₃ | 5-fluoropyridin-3-yl |
| 472 | CCH₃ | CH | H | 6-chloropyridin-3-yl |
| 473 | CCH₃ | CH | COCH₃ | 6-chloropyridin-3-yl |
| 474 | CCH₃ | CH | CH₃ | 6-chloropyridin-3-yl |
| 475 | CCH₃ | CH | H | 5-chloropyridin-3-yl |
| 476 | CCH₃ | CH | COCH₃ | 5-chloropyridin-3-yl |
| 477 | CCH₃ | CH | CH₃ | 5-chloropyridin-3-yl |
| 478 | CCH₃ | CH | H | isothiazole-4-yl |
| 479 | CCH₃ | CH | COCH₃ | isothiazole-4-yl |
| 480 | CCH₃ | CH | CH₃ | isothiazole-4-yl |
| 481 | CCH₃ | CCH₃ | H | phenyl |
| 482 | CCH₃ | CCH₃ | COCH₃ | phenyl |
| 483 | CCH₃ | CCH₃ | CH₃ | phenyl |
| 484 | CCH₃ | CCH₃ | H | 3-fluorophenyl |
| 485 | CCH₃ | CCH₃ | COCH₃ | 3-fluorophenyl |
| 486 | CCH₃ | CCH₃ | CH₃ | 3-fluorophenyl |
| 487 | CCH₃ | CCH₃ | H | 3,5-difluorophenyl |
| 488 | CCH₃ | CCH₃ | COCH₃ | 3,5-difluorophenyl |
| 489 | CCH₃ | CCH₃ | CH₃ | 3,5-difluorophenyl |
| 490 | CCH₃ | CCH₃ | H | 3-chlorophenyl |
| 491 | CCH₃ | CCH₃ | COCH₃ | 3-chlorophenyl |
| 492 | CCH₃ | CCH₃ | CH₃ | 3-chlorophenyl |
| 493 | CCH₃ | CCH₃ | H | 3,5-dichlorophenyl |
| 494 | CCH₃ | CCH₃ | COCH₃ | 3,5-dichlorophenyl |
| 495 | CCH₃ | CCH₃ | CH₃ | 3,5-dichlorophenyl |
| 496 | CCH₃ | CCH₃ | H | 3-methylphenyl |
| 497 | CCH₃ | CCH₃ | COCH₃ | 3-methylphenyl |
| 498 | CCH₃ | CCH₃ | CH₃ | 3-methylphenyl |
| 499 | CCH₃ | CCH₃ | H | 3,5-dimethylphenyl |
| 500 | CCH₃ | CCH₃ | COCH₃ | 3,5-dimethylphenyl |
| 501 | CCH₃ | CCH₃ | CH₃ | 3,5-dimethylphenyl |
| 502 | CCH₃ | CCH₃ | H | 3-methoxyphenyl |
| 503 | CCH₃ | CCH₃ | COCH₃ | 3-methoxyphenyl |
| 504 | CCH₃ | CCH₃ | CH₃ | 3-methoxyphenyl |
| 505 | CCH₃ | CCH₃ | H | 3,5-dimethoxyphenyl |
| 506 | CCH₃ | CCH₃ | COCH₃ | 3,5-dimethoxyphenyl |
| 507 | CCH₃ | CCH₃ | CH₃ | 3,5-dimethoxyphenyl |
| 508 | CCH₃ | CCH₃ | H | pyridin-4-yl |
| 509 | CCH₃ | CCH₃ | COCH₃ | pyridin-4-yl |
| 510 | CCH₃ | CCH₃ | CH₃ | pyridin-4-yl |
| 511 | CCH₃ | CCH₃ | H | 2-fluoropyridin-4-yl |
| 512 | CCH₃ | CCH₃ | COCH₃ | 2-fluoropyridin-4-yl |
| 513 | CCH₃ | CCH₃ | CH₃ | 2-fluoropyridin-4-yl |
| 514 | CCH₃ | CCH₃ | H | 2,6-difluoropyridin-4-yl |
| 515 | CCH₃ | CCH₃ | COCH₃ | 2,6-difluoropyridin-4-yl |
| 516 | CCH₃ | CCH₃ | CH₃ | 2,6-difluoropyridin-4-yl |
| 517 | CCH₃ | CCH₃ | H | 2-chloropyridin-4-yl |
| 518 | CCH₃ | CCH₃ | COCH₃ | 2-chloropyridin-4-yl |
| 519 | CCH₃ | CCH₃ | CH₃ | 2-chloropyridin-4-yl |
| 520 | CCH₃ | CCH₃ | H | 2,6-dichloropyridin-4-yl |
| 521 | CCH₃ | CCH₃ | COCH₃ | 2,6-dichloropyridin-4-yl |
| 522 | CCH₃ | CCH₃ | CH₃ | 2,6-dichloropyridin-4-yl |
| 523 | CCH₃ | CCH₃ | H | pyridin-3-yl |
| 524 | CCH₃ | CCH₃ | COCH₃ | pyridin-3-yl |
| 525 | CCH₃ | CCH₃ | CH₃ | pyridin-3-yl |
| 526 | CCH₃ | CCH₃ | H | 6-fluoropyridin-3-yl |
| 527 | CCH₃ | CCH₃ | COCH₃ | 6-fluoropyridin-3-yl |
| 528 | CCH₃ | CCH₃ | CH₃ | 6-fluoropyridin-3-yl |
| 529 | CCH₃ | CCH₃ | H | 5-fluoropyridin-3-yl |
| 530 | CCH₃ | CCH₃ | COCH₃ | 5-fluoropyridin-3-yl |
| 531 | CCH₃ | CCH₃ | CH₃ | 5-fluoropyridin-3-yl |
| 532 | CCH₃ | CCH₃ | H | 6-chloropyridin-3-yl |
| 533 | CCH₃ | CCH₃ | COCH₃ | 6-chloropyridin-3-yl |
| 534 | CCH₃ | CCH₃ | CH₃ | 6-chloropyridin-3-yl |
| 535 | CCH₃ | CCH₃ | H | 5-chloropyridin-3-yl |
| 536 | CCH₃ | CCH₃ | COCH₃ | 5-chloropyridin-3-yl |
| 537 | CCH₃ | CCH₃ | CH₃ | 5-chloropyridin-3-yl |
| 538 | CCH₃ | CCH₃ | H | isothiazole-4-yl |
| 539 | CCH₃ | CCH₃ | COCH₃ | isothiazole-4-yl |
| 540 | CCH₃ | CCH₃ | CH₃ | isothiazole-4-yl |

wherein
a) 540 compounds of formula (I.a):

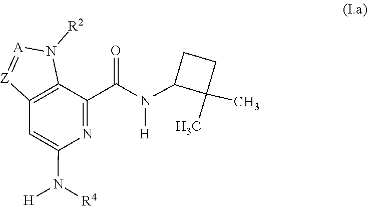

wherein A, Z, R² and R⁴ are as defined in Table 1.
b) 540 compounds of formula (I.b):

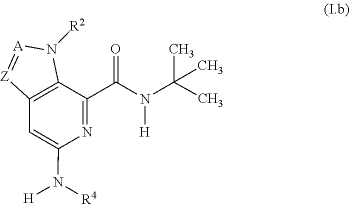

wherein A, Z, R² and R⁴ are as defined in Table 1.
c) 540 compounds of formula (I.c):

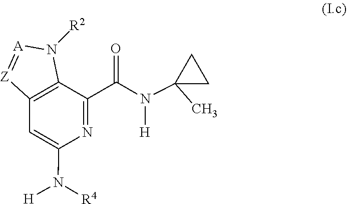

wherein A, Z, R² and R⁴ are as defined in Table 1.
d) 540 compounds of formula (I.d):

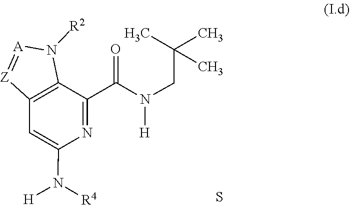

wherein A, Z, R² and R⁴ are as defined in Table 1.
e) 540 compounds of formula (I.e):

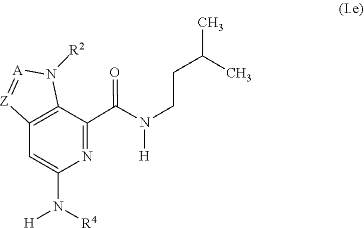

wherein A, Z, R² and R⁴ are as defined in Table 1.
f) 540 compounds of formula (I.f):

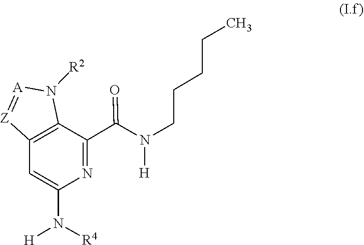

wherein A, Z, R² and R⁴ are as defined in Table 1.
g) 540 compounds of formula (I.g):

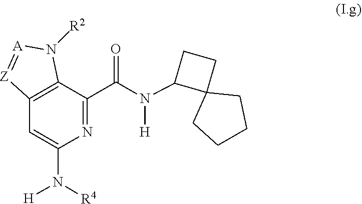

wherein A, Z, R² and R⁴ are as defined in Table 1.

TABLE 2
Individual compounds of formula (I) accordinq to the invention

| Cpd No. | A | Z | R¹ | R² | R⁴ |
|---|---|---|---|---|---|
| 001 | N | N | CN | H | 2,6-difluoropyridin-4-yl |
| 002 | N | N | CN | COCH₃ | 2,6-difluoropyridin-4-yl |
| 003 | N | N | COCH₃ | H | 2,6-difluoropyridin-4-yl |
| 004 | N | N | COCH₃ | COCH₃ | 2,6-difluoropyridin-4-yl |
| 005 | N | N | CN | H | 3,5-difluorophenyl |
| 006 | N | N | CN | COCH₃ | 3,5-difluorophenyl |
| 007 | N | N | COCH₃ | H | 3,5-difluorophenyl |
| 008 | N | N | COCH₃ | COCH₃ | 3,5-difluorophenyl |
| 009 | N | CH | CN | H | 2,6-difluoropyridin-4-yl |
| 010 | N | CH | CN | COCH₃ | 2,6-difluoropyridin-4-yl |
| 011 | N | CH | COCH₃ | H | 2,6-difluoropyridin-4-yl |
| 012 | N | CH | COCH₃ | COCH₃ | 2,6-difluoropyridin-4-yl |
| 013 | N | CH | CN | H | 3,5-difluorophenyl |
| 014 | N | CH | CN | COCH₃ | 3,5-difluorophenyl |
| 015 | N | CH | COCH₃ | H | 3,5-difluorophenyl |
| 016 | N | CH | COCH₃ | COCH₃ | 3,5-difluorophenyl |
| 017 | CH | N | CN | H | 2,6-difluoropyridin-4-yl |
| 018 | CH | N | CN | COCH₃ | 2,6-difluoropyridin-4-yl |
| 019 | CH | N | COCH₃ | H | 2,6-difluoropyridin-4-yl |
| 020 | CH | N | COCH₃ | COCH₃ | 2,6-difluoropyridin-4-yl |
| 021 | CH | N | CN | H | 3,5-difluorophenyl |
| 022 | CH | N | CN | COCH₃ | 3,5-difluorophenyl |
| 023 | CH | N | COCH₃ | H | 3,5-difluorophenyl |
| 024 | CH | N | COCH₃ | COCH₃ | 3,5-difluorophenyl |
| 025 | CH | CH | CN | H | 2,6-difluoropyridin-4-yl |
| 026 | CH | CH | CN | COCH₃ | 2,6-difluoropyridin-4-yl |
| 027 | CH | CH | COCH₃ | H | 2,6-difluoropyridin-4-yl |
| 028 | CH | CH | COCH₃ | COCH₃ | 2,6-difluoropyridin-4-yl |
| 029 | CH | CH | CN | H | 3,5-difluorophenyl |
| 030 | CH | CH | CN | COCH₃ | 3,5-difluorophenyl |
| 031 | CH | CH | COCH₃ | H | 3,5-difluorophenyl |
| 032 | CH | CH | COCH₃ | COCH₃ | 3,5-difluorophenyl |

wherein
h) 32 compounds of formula (I.h):

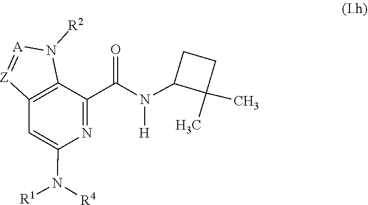

wherein A, Z, R¹, R² and R⁴ are as defined in Table 2.
j) 32 compounds of formula (I.j):

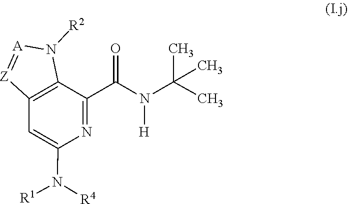

wherein A, Z, R¹, R² and R⁴ are as defined in Table 2.
k) 32 compounds of formula (I.k):

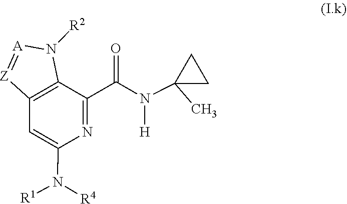

wherein A, Z, R¹, R² and R⁴ are as defined in Table 2.
m) 32 compounds of formula (I.m):

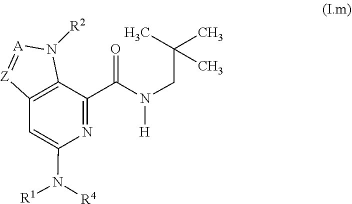

wherein A, Z, R¹, R² and R⁴ are as defined in Table 2.
n) 32 compounds of formula (I.n):

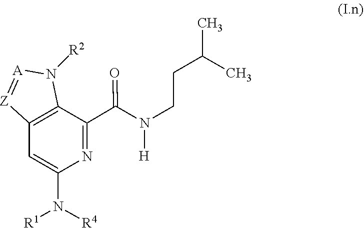

wherein A, Z, R¹, R² and R⁴ are as defined in Table 2.
o) 32 compounds of formula (I.o):

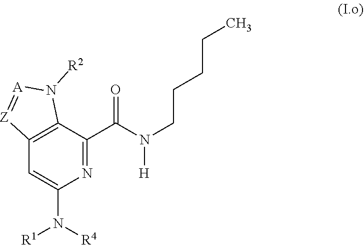

wherein A, Z, R¹, R² and R⁴ are as defined in Table 2.
p) 32 compounds of formula (I.p):

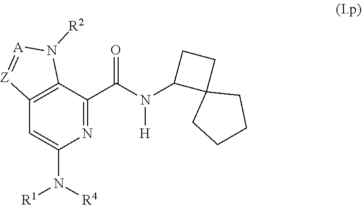

wherein A, Z, R¹, R² and R⁴ are as defined in Table 2.

### FORMULATION EXAMPLES

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

Emulsifiable concentrate

| | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

Extruder dranules

| | |
|---|---|
| Active ingredient [compound of formula (I)] | 15% |
| sodium lignosulfonate | 2% |
| carboxymethylcellulose | 1% |
| Kaolin | 82% |

The active ingredient is mixed and ground with the adjuvants, and the mixture is moistened with water. The mixture is extruded and then dried in a stream of air.

Coated granules

| | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

Suspension concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution in water) | 0.5% |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinyl alcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### EXAMPLES

The Examples which follow serve to illustrate the invention. The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

Compounds of formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

List of Abbreviations br. s=broad singlet, ° C.=degrees Celsius, CDCl₃=chloroform-d, d=doublet, dd=doublet of doublets, DIPEA=N,N-diisopropylethylamine, DMF=dimethylformamide, HATU=1-[Bis(dimethylamino) methylene]-1H-1,2,3-triazolo[4,5-b]pyridinium 3-oxid hexafluorophosphate, m=multiplet, MHz=mega hertz, s=singlet, THF=tetrahydrofuran

### Example 1: This example illustrates the preparation of 5-(3,5-difluoroanilino)-N-(2,2-dimethylcyclobutyl)-1H-pyrazolo[3,4-c]pyridine-7-carboxamide (Compound P-6)

### a) Preparation of 6-chloro-4-methyl-pyridin-3-amine

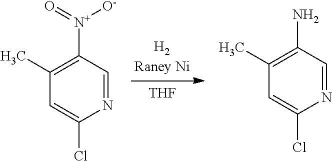

Raney Nickel (0.50 equiv.) was added portionwise to a solution of 2-chloro-4-methyl-5-nitro-pyridine (10.0 g, 57.9 mmol, 1.0 equiv.) in THE (290 mL). The suspension was purged with hydrogen and stirred at room temperature overnight. The reaction mixture was filtered over Celite and concentrated in vacuo. The desired 6-chloro-4-methyl-pyridin-3-amine (7.86 g, 55.1 mmol, 95% yield) was obtained after purification by chromatography on silica gel (eluent: mixtures cyclohexane/ethyl acetate). ¹H-NMR (400 MHz, CDCl₃): δ=2.16 (d, 3H), 3.64 (br s, 2H), 7.00 (s, 1H), 7.78 (s, 1H).

### b) Preparation of 6-chloro-2-iodo-4-methyl-pyridin-3-amine

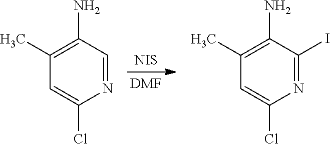

N-iodo succinimide (1.2 equiv.) was added portionwise to a stirred solution of 6-chloro-4-methyl-pyridin-3-amine (7.63 g, 53.5 mmol, 1.0 equiv.) in DMF (107 mL). The reaction mixture was stirred at room temperature overnight. The reaction mixture was diluted with ethyl acetate, washed three times with water, once with brine, dried over magnesium sulfate and concentrated in vacuo. The desired 6-chloro-2-iodo-4-methyl-pyridin-3-amine (8.01 g, 29.8 mmol, 56% yield) was obtained after purification by column chromatography on silica gel (eluent: mixtures cyclohexane/ethyl acetate). ¹H-NMR (400 MHz, CDCl₃): δ=2.21 (d, 3H), 4.07 (br s, 2H), 6.95 (s, 1H).

### c) Preparation of 3-amino-6-chloro-4-methyl-pyridine-2-carbonitrile

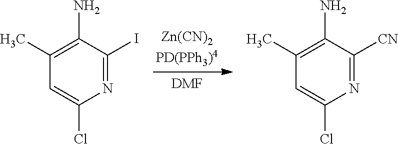

Under argon atmosphere, tetrakis(triphenylphosphine)palladium(0) (0.050 equiv.) was added to a degassed, stirred mixture of 6-chloro-2-iodo-4-methyl-pyridin-3-amine (8.01 g, 29.8 mmol, 1.0 equiv.) and zinc cyanide (1.0 equiv.) in DMF (119 mL). The reaction mixture was stirred at 80° C. for 4 hours. Then the reaction was cooled to room temperature, diluted with ethyl acetate and washed with saturated aqueous sodium bicarbonate. The water phase was extracted three times with ethyl acetate. The combined organic layers were washed with brine, dried over magnesium sulfate and concentrated in vacuo. The desired 3-amino-6-chloro-4-methyl-pyridine-2-carbonitrile (4.42 g, 26.4 mmol, 88% yield) was obtained after purification by column chromatography on silica gel (eluent: mixtures cyclohexane/ethyl acetate). ¹H-NMR (400 MHz, CDCl₃): δ=2.24 (d, 3H), 4.44 (br s, 2H), 7.19 (s, 1H).

### d) Preparation of N-acetyl-N-(6-chloro-2-cyano-4-methyl-3-pyridyl)acetamide

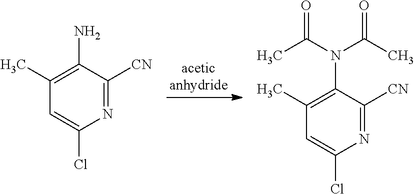 [SMILES: Cc1cc(Cl)nc(C#N)c1N ; "aceticanhydride" ; CC(=O)N(C(C)=O)c1c(C)cc(Cl)nc1C#N]

A mixture of 3-amino-6-chloro-4-methyl-pyridine-2-carbonitrile (4.42 g, 26.4 mmol, 1.0 equiv.) in acetic anhydride (50 equiv.) was stirred at 130° C. for 3 days. The reaction mixture was cooled down to room temperature and concentrated in vacuo to afford crude N-acetyl-N-(6-chloro-2-cyano-4-methyl-3-pyridyl)acetamide (6.49 g, 25.8 mmol). ¹H-NMR (400 MHz, CDCl₃): δ=2.27 (d, 3H), 2.37 (s, 6H), 7.55 (d, 1H).

### e) Preparation of N-(6-chloro-2-cyano-4-methyl-3-pyridyl)acetamide

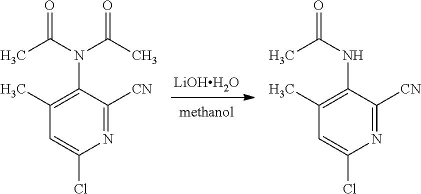 [SMILES: CC(=O)N(C(C)=O)c1c(C)cc(Cl)nc1C#N ; "LiOH·H2O" ; CC(=O)Nc1c(C)cc(Cl)nc1C#N]

Lithium hydroxide monohydrate (5.0 equiv.) was added to a stirred solution of crude N-acetyl-N-(6-chloro-2-cyano-4-methyl-3-pyridyl)acetamide (6.49 g, 25.8 mmol, 1.0 equiv.) in methanol (250 mL). The reaction mixture was stirred at room temperature for 2 hours and then concentrated in vacuo. The residue was diluted in water and extracted three times with ethyl acetate. The combined organic layers were dried over magnesium sulfate and concentrated in vacuo. The desired N-(6-chloro-2-cyano-4-methyl-3-pyridyl)acetamide (2.87 g, 13.7 mmol, 53% yield) was obtained after purification by column chromatography on silica gel (eluent: mixtures cyclohexane/ethyl acetate). ¹H-NMR (400 MHz, CDCl₃): δ=2.27-2.36 (m, 6H), 7.40 (br s, 1H), 7.45 (s, 1H).

### f) Preparation of 1-acetyl-5-chloro-pyrazolo[3,4-c]pyridine-7-carbonitrile

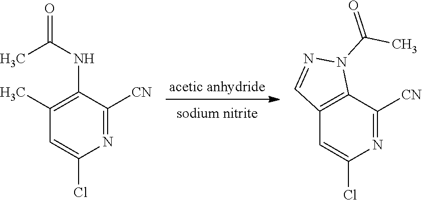

Sodium nitrite (4.5 equiv.) was added to a mixture of N-(6-chloro-2-cyano-4-methyl-3-pyridyl)acetamide (2.87 g, 13.7 mmol, 1.0 equiv.) and acetic anhydride (65 mL). The reaction mixture was stirred at room temperature for 1 hour, then at 50° C. for 4 hours. The reaction mixture was cooled to room temperature and poured into ice-water. The resulting precipitate was collected and dried over vacuo to afford the desired 1-acetyl-5-chloro-pyrazolo[3,4-c]pyridine-7-carbonitrile (2.58 g, 11.7 mmol, 85% yield). ¹H-NMR (400 MHz, CDCl₃): δ=2.86 (s, 3H), 7.96 (s, 1H), 8.30 (s, 1H).

### g) Preparation of 5-chloro-1H-pyrazolo[3,4-c]pyridine-7-carboxylic Acid

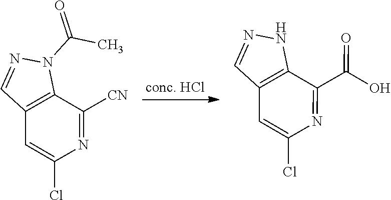

A mixture of 1-acetyl-5-chloro-pyrazolo[3,4-c]pyridine-7-carbonitrile (2.58 g, 11.7 mmol, 1.0 equiv.) and concentrated hydrochloric acid (37% in H₂O, 50 equiv.) was stirred at 100° C. overnight. The reaction mixture was cooled to room temperature and concentrated in vacuo. Direct purification of the crude by chromatography on silica gel (eluent: mixtures ethyl acetate/methanol) afforded the desired 5-chloro-1H-pyrazolo[3,4-c]pyridine-7-carboxylic acid (1.53 g, 7.74 mmol, 66% yield). ¹H-NMR (400 MHz, DMSO-d6): δ=8.18 (s, 1H), 8.34 (s, 1H), 13.83 (br s, 1H) 13.89-14.28 (m, 1H).

### h) Preparation of 5-chloro-N-(2,2-dimethylcyclobutyl)-1H-pyrazolo[3,4-c]pyridine-7-carboxamide

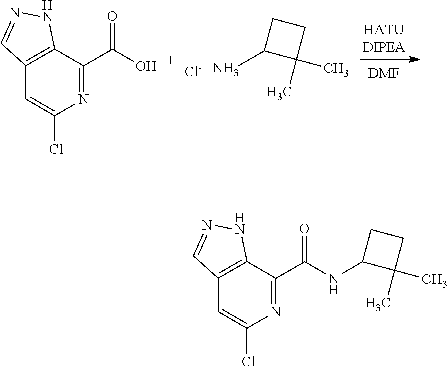

To a solution of 5-chloro-1H-pyrazolo[3,4-c]pyridine-7-carboxylic acid (0.500 g, 2.53 mmol, 1.0 equiv.) and 2,2-dimethylcyclobutylamine hydrochloride (1.1 equiv.) in DMF (25 ml), DIPEA (2.6 equiv.) and HATU (1.1 equiv.) were added in sequence. The reaction was stirred at room temperature for 2 hours. Then the reaction was quenched with saturated aqueous sodium bicarbonate and diluted with water. The water phase was extracted three times with ethyl acetate. The combined organic layers were washed with brine, dried over magnesium sulfate and concentrated in vacuo. The desired 5-chloro-N-(2,2-dimethylcyclobutyl)-1H-pyrazolo[3,4-c]pyridine-7-carboxamide (0.584 g, 2.10 mmol, 83% yield) was obtained after purification by column chromatography on silica gel (eluent: mixtures cyclohexane/ethyl acetate). ¹H-NMR (400 MHz, CDCl₃): δ=1.17 (s, 3H), 1.25 (s, 3H), 1.67 (m, 2H), 2.03 (m, 1H), 2.35 (dtd, 1H), 4.37 (q, 1H), 7.84 (d, 1H), 7.88 (br d, 1H), 8.15 (d, 1H), 11.79 (br s, 1H).

### i) Preparation of 5-(3,5-difluoroanilino)-N-(2,2-dimethylcyclobutyl)-1H-pyrazolo[3,4-c]pyridine-7-carboxamide

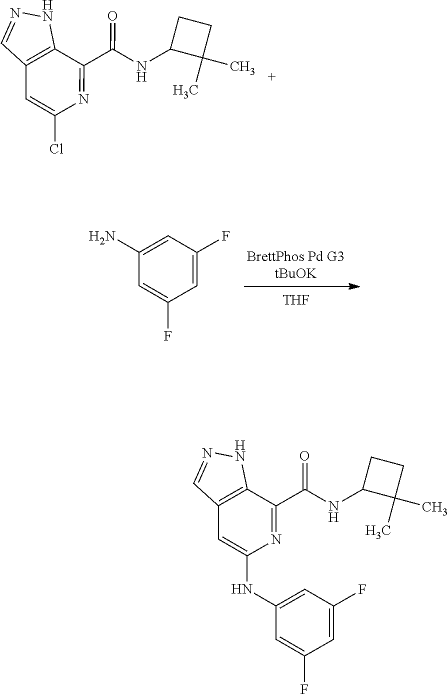 [SMILES: CC1(C)CCC1NC(=O)c1nc(Cl)cc2cn[nH]c12 ; Nc1cc(F)cc(F)c1 ; "THF" ; CC1(C)CCC1NC(=O)c1nc(Nc2cc(F)cc(F)c2)cc2cn[nH]c12]

Under argon atmosphere, BrettPhos-G3-palladacycle ([(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate, 0.10 equiv.) and potassium tert-butoxide (2.5 equiv.) were added to a degassed, stirred mixture of 5-chloro-N-(2,2-dimethylcyclobutyl)-1H-pyrazolo[3,4-c]pyridine-7-carboxamide (100 mg, 0.359 mmol, 1.0 equiv.) and 3,5-difluoro aniline (1.1 equiv.) in tetrahydrofuran (1.5 mL). The reaction was heated to 80° C. and stirred for 1 hour, then the mixture was cooled to room temperature. The volatiles were removed using a rotatory evaporator and the residue was dissolved in ethyl acetate. The organic phase was washed twice with water, dried over magnesium sulfate and concentrated in vacuo. Purification by column chromatography on silica gel (eluent: mixtures cyclohexane/ethyl acetate) afforded the desired 5-(3,5-difluoroanilino)-N-(2,2-dimethylcyclobutyl)-1H-pyrazolo[3,4-c]pyridine-7-carboxamide (82 mg, 62% yield). ¹H-NMR (400 MHz, CDCl₃): δ=1.18 (s, 3H), 1.23 (s, 3H), 1.66 (m, 2H), 1.97 (m, 1H), 2.36 (dd, 1H), 4.37 (d, 1H), 6.45 (m, 1H), 6.55 (s, 1H), 6.86 (dd, 2H), 7.40 (d, 1H), 7.86 (br d, 1H), 8.07 (d, 1H), 11.51 (br s, 1H).

### Example 2: This example illustrates the preparation of 5-[(2,6-difluoro-4-pyridyl)amino]-N-spiro[3.4]octan-3-yl-1H-pyrrolo[2,3-c]pyridine-7-carboxamide (Compound P-18)

### a) Preparation of 5,7-dichloro-1H-pyrrolo[2,3-c]pyridine

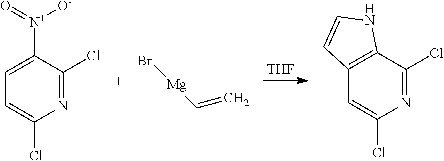

Under argon atmosphere, vinyl magnesium bromide (1 mol/L in THF, 3.5 equiv.) was added dropwise at −78° C. to a solution of 2,6-dichloro-3-nitro-pyridine (5.40 g, 28.0 mmol, 1.0 equiv.) in THE (170 mL). At the end of the addition, the reaction mixture was warmed up to −10° C. and stirred for 1 hour. Then the reaction mixture was quenched with saturated aqueous ammonium chloride and extracted three times with ethyl acetate. The combined organic layers were washed with brine, dried over magnesium sulfate and concentrated in vacuo. The desired 5,7-dichloro-1H-pyrrolo[2,3-c]pyridine (1.71 g, 9.14 mmol, 33% yield) was obtained after purification by column chromatography on silica gel (eluent: mixtures cyclohexane/ethyl acetate). ¹H-NMR (400 MHz, CDCl₃): δ=6.61 (dd, 1H), 7.48 (dd, 1H), 7.52 (d, 1H), 8.57 (br s, 1H).

### b) Preparation of 5-chloro-N-spiro[3.4]octan-3-yl-1H-pyrrolo[2,3-c]pyridine-7-carboxamide

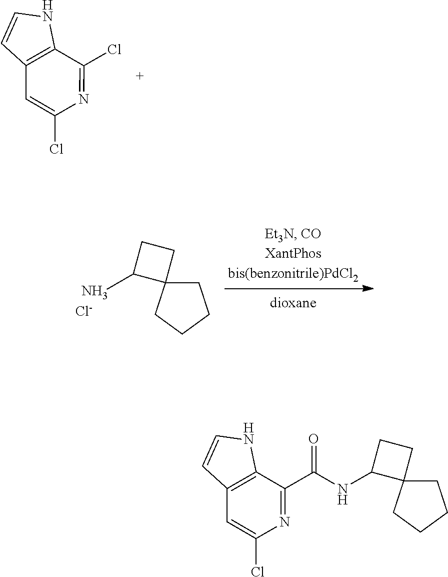 [SMILES: Clc1cc2cc[nH]c2c(Cl)n1 ; [Cl-] ; NC1CCC12CCCC2 ; O=C(NC1CCC12CCCC2)c1nc(Cl)cc2cc[nH]c12]

5,7-dichloro-1H-pyrrolo[2,3-c]pyridine (716 mg, 3.83 mmol, 1.0 equiv.), spiro[3.4]octan-3-amine; hydrochloride (1.5 equiv.), Xantphos (4,5-Bis(diphenylphosphino)-9,9-dimethylxanthene, 0.010 equiv.) and bis(benzonitrile)palladium(II) chloride (0.010 equiv.) were charged in an autoclave, which was then purged with argon. Then dioxane (23 mL) and triethylamine (3.0 equiv.) were added under argon to the autoclave. The reaction proceeded under 20 bar of carbon monoxide gas and at 130° C. overnight. Then the reaction mixture was cooled to room temperature and concentrated in vacuo. Direct purification of the crude by chromatography on silica gel (eluent: mixtures cyclohexane/ethyl acetate) afforded the desired 5-chloro-N-spiro[3.4]octan-3-yl-1H-pyrrolo[2,3-c]pyridine-7-carboxamide (600 mg, 1.97 mmol, 52% yield). ¹H-NMR (400 MHz, CDCl₃): δ=1.53-1.78 (m, 8H), 1.79-1.89 (m, 2H), 1.92-2.03 (m, 1H), 2.29-2.37 (m, 1H), 4.53 (q, 1H), 6.54 (dd, 1H), 7.52 (m, 1H), 7.68 (m, 1H), 8.03 (br d, 1H), 10.34 (br s, 1H).

### c) Preparation of 5-[(2,6-difluoro-4-pyridyl)amino]-N-spiro[3.4]octan-3-yl-1H-pyrrolo[2,3-c]pyridine-7-carboxamide

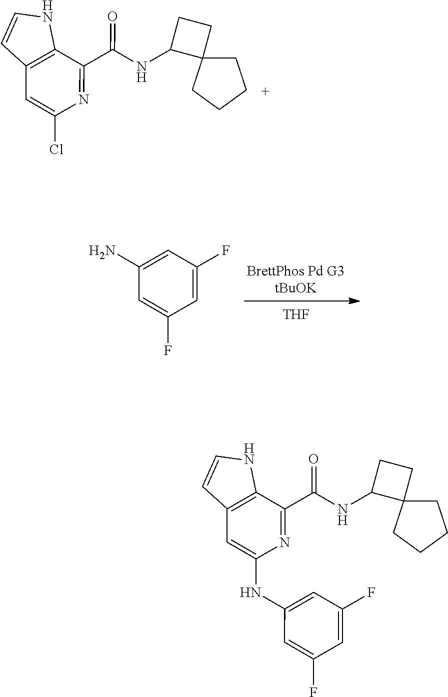 [SMILES: O=C(NC1CCC12CCCC2)c1nc(Cl)cc2cc[nH]c12 ; Nc1cc(F)cc(F)c1 ; "THF" ; O=C(NC1CCC12CCCC2)c1nc(Nc2cc(F)cc(F)c2)cc2cc[nH]c12]

Under argon atmosphere, BrettPhos-G3-palladacycle ([(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate, 0.10 equiv.) and potassium tert-butoxide (2.5 equiv.) were added to a degassed, stirred mixture of 5-chloro-N-spiro[3.4]octan-3-yl-1H-pyrrolo[2,3-c]pyridine-7-carboxamide (100 mg, 0.329 mmol, 1.0 equiv.) and 3,5-difluoro pyridine (1.1 equiv.) in THE (1.5 mL). The reaction was heated to 80° C. and stirred for 1 hour, then the mixture was cooled to room temperature. The volatiles were removed using a rotatory evaporator and the residue was dissolved in ethyl acetate. The organic phase was washed twice with water, dried over magnesium sulfate and concentrated in vacuo. Purification by column chromatography on silica gel (eluent: mixtures cyclohexane/ethyl acetate) afforded the desired 5-[(2,6-difluoro-4-pyridyl)amino]-N-spiro[3.4]octan-3-yl-1H-pyrrolo[2,3-c]pyridine-7-carboxamide (28 mg, 0.070 mmol, 21% yield). ¹H-NMR (400 MHz, CDCl₃): δ=1.57-1.84 (m, 10H), 1.85-1.97 (m, 1H), 2.35 (m, 1H), 4.56 (q, 1H), 6.55 (dd, 1H), 6.67 (s, 2H), 6.89 (s, 1H), 7.37 (s, 1H), 7.53 (t, 1H), 7.98 (br d, 1H), 10.25 (br s, 1H).

Throughout this description, temperatures are given in degrees Celsius (° C.) and “m.p.” means melting point. LC/MS means Liquid Chromatography Mass Spectrometry and the description of the apparatus and the method is: (Method A: ACQUITY UPLC from Waters, Waters UPLC HSS T3, 1.8 m particle size, 30×2.1 mm column, 0.85 mL/min., 60° C., H₂O/MeOH 95:5+0.05% HCOOH (90%)/CH₃CN+0.05% HCOOH (10%) −1.2 min.−CH₃CN+0.05% HCOOH (100%) −0.30 min., ACQUITY SQD Mass Spectrometer from Waters, ionization method: electrospray (ESI), Polarity: positive ions, Capillary (kV) 3.00, Cone (V) 30.00, Extractor (V) 2.00, Source Temperature (° C.) 150, Desolvation Temperature (° C.) 350, Cone Gas Flow (L/Hr) 0, Desolvation Gas Flow (L/Hr) 650). Method B: ACQUITY UPLC from Waters, Waters UPLC HSS T3, 1.8 μm particle size, 30×2.1 mm column, 0.85 mL/min., 60° C., H₂O/MeOH 95:5+0.05% HCOOH (90%)/CH₃CN+0.05% HCOOH (10%) −2.7 min.−CH₃CN+0.05% HCOOH (100%) −0.30 min., ACQUITY SQD Mass Spectrometer from Waters, ionization method: electrospray (ESI), Polarity: positive ions, Capillary (kV) 3.00, Cone (V) 30.00, Extractor (V) 2.00, Source Temperature (° C.) 150, Desolvation Temperature (° C.) 350, Cone Gas Flow (L/Hr) 0, Desolvation Gas Flow (L/Hr) 650). Method C: ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30 x 2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.).

TABLE 3
Melting point and LC/MS data (Rₜ = Retention time) for selected compounds of Table 1
and Table 2.

| | Compound | | Mp | |
|---|---|---|---|---|
| No. | Name | Structure | (° C.) | LC/MS |
| P-1 | 5-(3,5-difluoroanilino)- N-(2,2- dimethylcyclobutyl)- 1H-pyrrolo[2,3- c]pyridine-7- carboxamide | 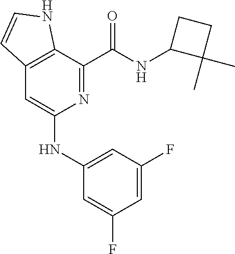 [SMILES: CC1(C)CCC1NC(=O)c1nc(Nc2cc(F)cc(F)c2)cc2cc[nH]c12] | 65-89 | Rₜ = 1.23 min (A); MS: m/z = 371 (M + 1) |
| P-2 | 5-[(2,6-difluoro-4- pyridyl)amino]-N-(2,2- dimethylcyclobutyl)- 1H-pyrrolo[2,3- c]pyridine-7- carboxamide | 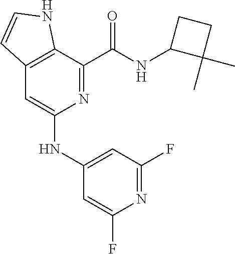 [SMILES: CC1(C)CCC1NC(=O)c1nc(Nc2cc(F)nc(F)c2)cc2cc[nH]c12] | 115-148 | Rₜ = 1.15 min (A); MS: m/z = 372 (M + 1) |
| P-3 | N-(2,2- dimethylcyclobutyl)-5- (thiadiazol-5- ylamino)-1H- pyrazolo[3,4- c]pyridine-7- carboxamide | 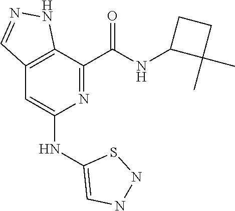 [SMILES: CC1(C)CCC1NC(=O)c1nc(NC2=CNNS2)cc2cn[nH]c12] | 178-195 | Rₜ = 0.86 min (A); MS: m/z = 344 (M + 1) |
| P-4 | N-(2,2- dimethylcyclobutyl)-5- [(1-methylpyrazol-4- yl)amino]-1H- pyrazolo[3,4- c]pyridine-7- carboxamide | 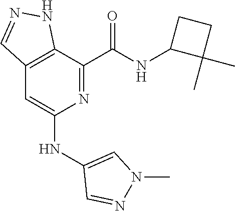 [SMILES: Cn1cc(Nc2cc3cn[nH]c3c(C(=O)NC3CCC3(C)C)n2)cn1] | 158-166 | Rₜ = 0.85 min (A); MS: m/z = 340 (M + 1) |
| P-5 | N-(2,2- dimethylcyclobutyl)-5- (isothiazol-4-ylamino)- 1H-pyrazolo[3,4- c]pyridine-7- carboxamide | 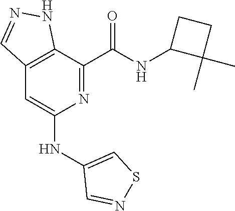 [SMILES: CC1(C)CCC1NC(=O)c1nc(Nc2cnsc2)cc2cn[nH]c12] | 158-166 | Rₜ = 0.95 min (A); MS: m/z = 343 (M + 1) |
| P-6 | 5-(3,5-difluoroanilino)- N-(2,2- dimethylcyclobutyl)- 1H-pyrazolo[3,4- c]pyridine-7- carboxamide | 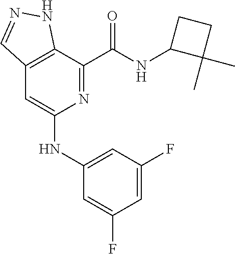 [SMILES: CC1(C)CCC1NC(=O)c1nc(Nc2cc(F)cc(F)c2)cc2cn[nH]c12] | 167-176 | Rₜ = 1.13 min (A); MS: m/z = 372 (M + 1) |
| P-7 | N-(2,2- dimethylcyclobutyl)-5- [(2-fluoro-4- pyridyl)amino]-1H- pyrazolo[3,4- c]pyridine-7- carboxamide | 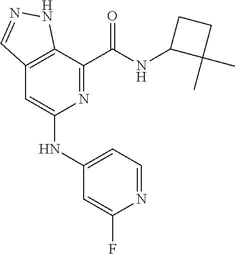 [SMILES: CC1(C)CCC1NC(=O)c1nc(Nc2ccnc(F)c2)cc2cn[nH]c12] | 222-238 | Rₜ = 0.95 min (A); MS: m/z = 355 (M + 1) |
| P-8 | 5-[(2,6-difluoro-4- pyridyl)amino]-N-(2,2- dimethylcyclobutyl)- 1H-pyrazolo[3,4- c]pyridine-7- carboxamide | 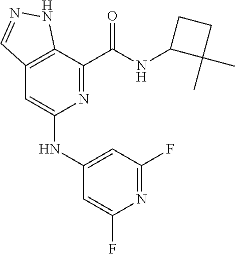 [SMILES: CC1(C)CCC1NC(=O)c1nc(Nc2cc(F)nc(F)c2)cc2cn[nH]c12] | 240-250 | Rₜ = 1.05 min (A); MS: m/z = 373 (M + 1) |
| P-9 | N-pentyl-5-(thiazol-2- ylamino)-1H- pyrazolo[3,4- c]pyridine-7- carboxamide | 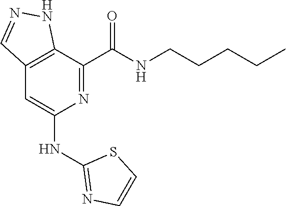 | | Rₜ = 0.90 min (A); MS: m/z = 321 (M + 1) |
| P-10 | 5-[(5-fluoro-3- pyridyl)amino]-N- pentyl-1H-pyrrolo[2,3- c]pyridine-7- carboxamide | 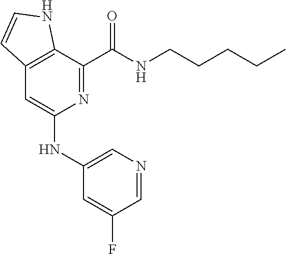 [SMILES: CCCCCNC(=O)c1nc(Nc2cncc(F)c2)cc2cc[nH]c12] | 158-162 | Rₜ = 1.01 min (A); MS: m/z = 342 (M + 1) |
| P-11 | 5-[(2,6-difluoro-4- pyridyl)amino]-N- pentyl-1H-pyrrolo[2,3- c]pyridine-7- carboxamide | 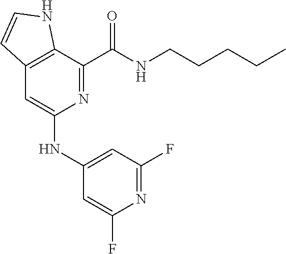 [SMILES: CCCCCNC(=O)c1nc(Nc2cc(F)nc(F)c2)cc2cc[nH]c12] | 220-233 | Rₜ = 1.09 min (A); MS: m/z = 360 (M + 1) |
| P-12 | N-spiro[3.4]octan-3- yl-5-(thiazol-2- ylamino)-1H- pyrazolo[3,4- c]pyridine-7- carboxamide | 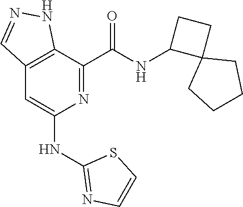 [SMILES: O=C(NC1CCC12CCCC2)c1nc(Nc2nccs2)cc2cn[nH]c12] | | Rₜ = 1.00 min (A); MS: m/z = 369 (M + 1) |
| P-13 | 5-[(5-fluoro-3- pyridyl)amino]-N- pentyl-1H- pyrazolo[3,4- c]pyridine-7- carboxamide | 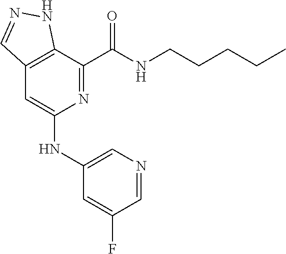 [SMILES: CCCCCNC(=O)c1nc(Nc2cncc(F)c2)cc2cn[nH]c12] | 155-168 | Rₜ = 0.93 min (A); MS: m/z = 343 (M + 1) |
| P-14 | 5-[(2,6-difluoro-4- pyridyl)amino]-N- pentyl-1H- pyrazolo[3,4- c]pyridine-7- carboxamide | 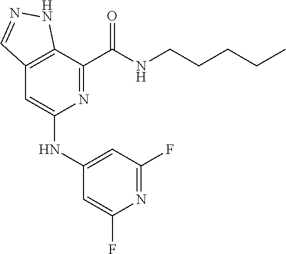 [SMILES: CCCCCNC(=O)c1nc(Nc2cc(F)nc(F)c2)cc2cn[nH]c12] | | Rₜ = 1.01 min (A); MS: m/z = 361 (M + 1) |
| P-15 | 5-[(5-fluoro-3- pyridyl)amino]-N- spiro[3.4]octan-3-yl- 1H-pyrazolo[3,4- c]pyridine-7- carboxamide | 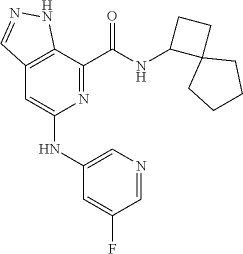 [SMILES: O=C(NC1CCC12CCCC2)c1nc(Nc2cncc(F)c2)cc2cn[nH]c12] | 185-195 | Rₜ = 1.03 min (A); MS: m/z = 381 (M + 1) |
| P-16 | N-spiro[3.4]octan-3- yl-5-(thiazol-2- ylamino)-1H- pyrrolo[2,3-c]pyridine- 7-carboxamide | 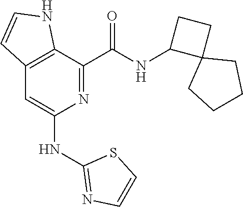 [SMILES: O=C(NC1CCC12CCCC2)c1nc(Nc2nccs2)cc2cc[nH]c12] | | Rₜ = 1.05 min (A); MS: m/z = 368 (M + 1) |
| P-17 | 5-[(5-fluoro-3- pyridyl)amino]-N- spiro[3.4]octan-3-yl- 1H-pyrrolo[2,3- c]pyridine-7- carboxamide | 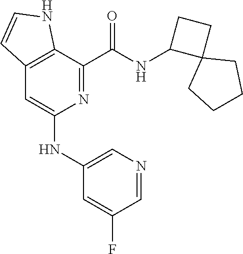 [SMILES: O=C(NC1CCC12CCCC2)c1nc(Nc2cncc(F)c2)cc2cc[nH]c12] | 180-191 | Rₜ = 1.10 min (A); MS: m/z = 380 (M + 1) |
| P-18 | 5-[(2,6-difluoro-4- pyridyl)amino]-N- spiro[3.4]octan-3-yl- 1H-pyrrolo[2,3- c]pyridine-7- carboxamide | 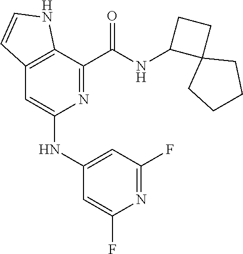 [SMILES: O=C(NC1CCC12CCCC2)c1nc(Nc2cc(F)nc(F)c2)cc2cc[nH]c12] | 170-178 | Rₜ = 1.17 min (A); MS: m/z = 398 (M + 1) |
| P-19 | 5-[(2,6-difluoro-4- pyridyl)amino]-N- spiro[3.4]octan-3-yl- 1H-pyrazolo[3,4- c]pyridine-7- carboxamide | 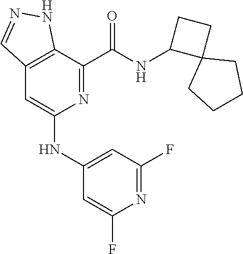 [SMILES: O=C(NC1CCC12CCCC2)c1nc(Nc2cc(F)nc(F)c2)cc2cn[nH]c12] | 168-180 | Rₜ = 1.09 min (A); MS: m/z = 399 (M + 1) |

### BIOLOGICAL EXAMPLES

### Example B1: Glomerella lagenarium (Colletotrichum lagenarium)/Liquid Culture (Anthracnose)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is measured photometrically 3-4 days after application. The following compounds gave at least 80% control of Glomerella lagenarium at 20 ppm when compared to untreated control under the same conditions, which showed extensive disease development: P-1, P-5, P-6, P-7, P-8, P-19.

### Example B2: Monographella nivalis (Microdochium nivale)/Liquid Culture (Foot Rot Cereals)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.
The following compounds gave at least 80% control of Monographella nivalis at 20 ppm when compared to untreated control under the same conditions, which showed extensive disease development: P-1, P-5, P-6, P-7, P-8, P-19.

### Example B3: Magnaporthe grisea (Pyricularia oryzae)/Rice/Leaf Disc Preventative (Rice Blast)

Rice leaf segments cv. Ballila are placed on agar in a multiwell plate (24-well format) and sprayed with the formulated test compound diluted in water. The leaf segments are inoculated with a spore suspension of the fungus 2 days after application. The inoculated leaf segments are incubated at 22° C. and 80% rh under a light regime of 24 h darkness followed by 12 h light/12 h darkness in a climate cabinet and the activity of a compound is assessed as percent disease control compared to untreated when an appropriate level of disease damage appears in untreated check leaf segments (5-7 days after application). The following compounds gave at least 80% control of Magnaporthe grisea at 200 ppm when compared to untreated control under the same conditions, which showed extensive disease development: P-6, P-7, P-8.

### Example B4: Pyrenophora teres/Barley/Leaf Disc Preventative (Net Blotch)

Barley leaf segments cv. Hasso are placed on agar in a multiwell plate (24-well format) and sprayed with the formulated test compound diluted in water. The leaf segments are inoculated with a spore suspension of the fungus 2 days after application. The inoculated leaf segments are incubated at 20° C. and 65% rh under a light regime of 12 h light/12 h darkness in a climate cabinet and the activity of a compound is assessed as disease control compared to untreated when an appropriate level of disease damage appears in untreated check leaf segments (5-7 days after application). The following compounds gave at least 80% control of Pyrenophora teres at 200 ppm when compared to untreated control under the same conditions, which showed extensive disease development: P-6, P-8.

### Example B5: Mycosphaerella graminicola (Septoria tritici)/Liquid Culture (Septoria blotch)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application. The following compounds gave at least 80% control of Mycosphaerella graminicola at 20 ppm when compared to untreated control under the same conditions, which showed extensive disease development: P-1, P-2, P-5, P-6, P-7, P-8, P-18, P-19.

1. A compound of formula (I): 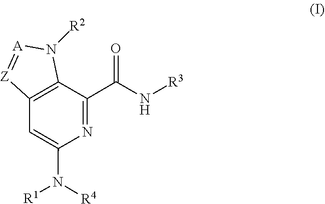
wherein
A is N or C—R⁵;
Z is N or C—R⁵;
R¹ is hydrogen, cyano, formyl, C₁-C₆alkylcarbonyl, C₁-C₆alkoxycarbonyl, C₁-C₆haloalkylcarbonyl, C₁-C₆alkoxyC₁-C₆alkylcarbonyl, C₃-C₆cycloalkylcarbonyl, C₁-C₆alkoxyC₁-C₃alkoxycarbonyl, C₁-C₆alkoxyoxalyl, C₁-C₆alkoxycarbonylC₁-C₄alkylC₁-C₆alkoxycarbonyl, C₁-C₆alkylsulfanylcarbonyl, or phenylcarbonyl;
R² is hydrogen, C₁-C₆alkyl, C₁-C₆haloalkyl, cyano, formyl, C₁-C₆alkylcarbonyl, C₁-C₆alkoxycarbonyl, C₁-C₆haloalkylcarbonyl, C₁-C₆alkoxyC₁-C₆alkylcarbonyl, C₃-C₆cycloalkylcarbonyl, C₁-C₆alkoxyC₁-C₃alkoxycarbonyl, C₁-C₆alkoxyoxalyl, C₁-C₆alkoxycarbonylC₁-C₄alkylC₁-C₆alkoxycarbonyl, C₂-C₆alkenyloxycarbonyl, C₂-C₆alkynyloxycarbonyl, C₁-C₆alkylsulfanylcarbonyl, or phenylcarbonyl;
R³ is C₁-C₈alkyl, C₁-C₈haloalkyl, C₁-C₈alkoxy, C₃-C₈cycloalkyl, C₃-C₈cycloalkylC₁-C₂alkyl, wherein the cycloalkyl groups are optionally substituted with 1 to 3 groups represented by R⁶, phenyl, phenylC₁-C₂alkyl, heteroaryl, heteroarylC₁-C₂alkyl, wherein the heteroaryl is a 5-or 6-membered aromatic monocyclic ring comprising 1, 2, 3 or 4 heteroatoms individually selected from nitrogen, oxygen and sulfur, heterocyclyl, heterocyclylC₁-C₂alkyl, wherein the heterocyclyl is a 4-, 5-or 6-membered non-aromatic monocyclic ring comprising 1, 2 or 3 heteroatoms individually selected from nitrogen, oxygen and sulfur, or a 5-to 10-membered non-aromatic spirocyclic carbobi- or carbotri-cyclyl ring system optionally comprising 1, 2, 3, 4 or 5 heteroatoms individually selected from nitrogen, oxygen and sulfur, and optionally bonded to the rest of the molecule through a C₁-C₂alkylene linker;
R⁴ is phenyl or heteroaryl, wherein heteroaryl is a 5-or 6-membered aromatic monocyclic ring comprising 1, 2 or 3 heteroatoms individually selected from nitrogen, oxygen and sulfur, and wherein the phenyl or heteroaryl group is optionally substituted by 1, 2 or 3 substituents, which may be the same or different, selected from R⁷;
R⁵ is hydrogen, halogen, or C₁-C₄alkyl;
R⁶ is halogen, C₁-C₄alkyl, C₁-C₄alkoxy, or C₁-C₄haloalkyl; and
R⁷ is halogen, cyano, C₁-C₄alkyl, C₁-C₄haloalkyl, C₁-C₄alkoxy, or C₁-C₄haloalkoxy;
or a salt or an N-oxide thereof.

2. The compound according to claim 1, wherein A is N or CH.

3. The compound according to claim 1, wherein Z is CH.

4. The compound according to claim 1, wherein R¹ is hydrogen, cyano, or C₁-C₆alkylcarbonyl.

5. The compound according to claim 1, wherein R² is hydrogen, C₁-C₄alkyl, or C₁-C₄alkylcarbonyl.

6. The compound according to claim 1, wherein R³ is C₁-C₈alkyl, C₁-C₆haloalkyl, C₁-C₈alkoxy, C₃-C₆cycloalkyl, C₃-C₆cycloalkylC₁-C₂alkyl, wherein the cycloalkyl groups are optionally substituted with 1 or 2 groups represented by R⁶, phenyl, heteroaryl, wherein the heteroaryl is a 5-or 6-membered aromatic monocyclic ring comprising 1, 2, or 3 heteroatoms individually selected from nitrogen, oxygen and sulfur, heterocyclyl, wherein the heterocyclyl is a 5-or 6-membered non-aromatic monocyclic ring comprising 1 or 2 heteroatoms individually selected from nitrogen, oxygen and sulfur, or a 6-to 10-membered non-aromatic spirocyclic carbobi-cyclyl ring system optionally comprising 1, or 2 heteroatoms individually selected from nitrogen, oxygen and sulfur.

7. The compound according to claim 1, wherein R³ is t-butyl, n-pentyl, isopentyl, 2,2-dimethylpropyl, 1-methylcyclopropyl, 2,2-dimethylcyclobutyl, or spiro[3.4]octan-3-yl.

8. The compound according to claim 1, wherein R⁴ is phenyl or heteroaryl, wherein heteroaryl is a 5-or 6-membered aromatic monocyclic ring comprising 1, 2 or 3 heteroatoms individually selected from nitrogen and sulfur, and wherein the phenyl or heteroaryl group is optionally substituted by 1 or 2 substituents, which may be the same or different, selected from R⁷.

9. The compound according to claim 1, wherein R⁴ is phenyl, pyridyl, isothiazolyl, thiadiazolyl, or pyrazolyl, wherein each phenyl, pyridyl, isothiazolyl, thiadiazolyl, or pyrazolyl moiety is optionally substituted by 1 or 2 substituents, which may be the same or different, selected from R⁷.

10. The compound according to claim 1, wherein R⁶ is methyl.

11. The compound according to claim 1, wherein R⁷ is chloro, fluoro, methyl, or methoxy.

12. An agrochemical composition comprising a fungicidally effective amount of a compound of formula (I) according to claim 1.

13. The composition according to claim 12, further comprising at least one additional active ingredient and/or an agrochemically-acceptable diluent or carrier.

14. A method of controlling or preventing infestation of useful plants by phytopathogenic microorganisms, wherein a fungicidally effective amount of a compound of formula (I) according to claim 1, is applied to the plants, to parts thereof or the locus thereof.

15. Use of a compound of formula (I) according to claim 1 as a fungicide.

16. The compound according to claim 2, wherein Z is CH.

17. A method of controlling or preventing infestation of useful plants by phytopathogenic microorganisms, wherein a fungicidally effective amount of a compound of formula (I) according to a composition according to claim 12, is applied to the plants, to parts thereof or the locus thereof.
